(19) United States
(12) Patent Application Publication
(10) Pub. No.: US 2017/0281646 A1
(43) Pub. Date: Oct. 5, 2017

(54) STEROID HORMONE PHARMACEUTICAL COMPOSITION

(71) Applicant: TherapeuticsMD, Inc., Boca Raton, FL (US)

(72) Inventors: PHILIP B. INSKEEP, East Lyme, CT (US); PETER H.R. PERSICANER, Boca Raton, FL (US); ANNETTE SHADIACK, Somerset, NJ (US); THORSTEINN THORSTEINSSON, Boynton Beach, FL (US); FREDERICK D. SANCILIO, Palm Beach Gardens, FL (US); JASON D. LEGASSIE, Stuart, FL (US)

(21) Appl. No.: 15/475,052

(22) Filed: Mar. 30, 2017

Related U.S. Application Data
(60) Provisional application No. 62/317,359, filed on Apr. 1, 2016.

(51) Int. Cl. A61K 31/57; A61K 9/107; A61K 47/26; A61K 47/06; A61K 31/565; A61K 47/44

(52) U.S. Cl. CPC A61K 31/57; A61K 31/565; A61K 47/44; A61K 47/26; A61K 47/06; A61K 9/107

(57) ABSTRACT

This disclosure provides a novel pharmaceutical composition for delivering steroid hormones, such as estradiol and progesterone, to a patient in need thereof.

### CROSS-REFERENCES TO RELATED APPLICATIONS

This application claims priority to U.S. Provisional Patent Application No. 62/317,359, filed on Apr. 1, 2016, which application is incorporated herein by reference in its entirety.

### FIELD

This disclosure relates to the field of steroid hormones and in particular, provides a pharmaceutical composition comprising steroid hormones, such as estradiol and progesterone, having enhanced oral bioavailability compared with currently marketed formulations.

### BACKGROUND

Steroid hormones are vital constituents for the proper functioning of the human body and can be classified into five groups based on the receptors to which they bind, namely: glucocorticoids, mineralocorticoids, androgens, estrogens, and progestogens. It is known that steroid hormones aid in regulating metabolism, regulating water and salt function, regulating immune function, controlling inflammation, and developing sexual characteristics.

Despite their wide ranging biological activity, steroid hormones are difficult to deliver to a subject experiencing a disease or disorder where additional steroid hormone could help treat the disease or disorder. Hormone Replacement Therapy (HRT) is an example of a medical treatment that is designed to increase hormone levels in women who lack adequate hormone production in order to treat conditions and diseases associated with low estrogen levels and/or low progesterone levels. HRT can mitigate and prevent symptoms caused by diminished circulating estrogen and progesterone hormones in a pre-menopausal, peri-menopausal, menopausal, or post-menopausal subject. However, progesterone and estradiol have extremely poor oral bioavailability due to their respective limited water solubilities. As a result (and particularly with progesterone), when given orally they must be administered in a sufficiently high dose to obtain the desired pharmacokinetic profile. Higher dosages of progesterone, however, make co-formulating progesterone with estradiol very challenging, and higher dosages of progesterone are inherently less desirable as the greater the quantity dosed, the greater the likelihood that additional drug, beyond what the patient requires, could enter the body and exert an effect.

Progesterone (CAS#57-83-0), also known as P4 (pregn-4-ene-3,20-dione), is a C-21 steroid hormone involved in the female menstrual cycle, pregnancy, and embryogenesis of humans and other species. Progesterone belongs to a class of hormones called progestogens, and is the major naturally occurring, endogenous human progestogen. The use of progesterone and its analogues has many medical applications, both to address acute conditions as well as the long-term decline of natural progesterone levels. Undesirable side effects exist due to irregular, inconsistent, or decreased hormone production in pre-, peri-, menopausal, and post-menopausal females. Progesterone is indicated for use in the prevention of endometrial hyperplasia in non-hysterectomized postmenopausal women who are receiving systemic estrogen. Progesterone is also indicated for use in secondary amenorrhea.

Estradiol (CAS#50-28-2), also known as 17β-estradiol, oestradiol, or E2, is found endogenously in the human body and is the primary female sex hormone. Estradiol contributes to regulation of estrous and menstrual reproduction cycles in females, development of reproductive tissues, and maintenance of bone tissue, among other processes. Estradiol deficiency in female subjects is implicated in conditions such as preterm birth, sleep disturbances, mood changes, vulvo-vaginal atrophy, and osteoporosis.

Existing oral compositions are formulated such that high dosages of hormones or various synthetic estrogen and progesterone analogs are administered, and most oral progesterone compositions suffer from progesterone's limited absorption and bioavailability. Therefore, new oral compositions for more effective delivery of progesterone and estradiol are needed. The invention disclosed herein meets this and other needs.

### SUMMARY

This disclosure provides a pharmaceutical composition comprising estradiol, progesterone, at least one lipophilic surfactant, and at least hydrophilic surfactant, and, optionally, a terpene. In certain embodiments, the at least one lipophilic surfactant can comprise a first lipophilic surfactant and a second lipophilic surfactant. In some embodiments, the at least one hydrophilic surfactant can comprise a first hydrophilic surfactant and a second hydrophilic surfactant. In certain embodiments, the optional terpene can be a monocyclic terpene such as d-limonene. In certain embodiments, the pharmaceutical composition comprises bio-identical progesterone and bio-identical estradiol.

This disclosure further provides methods of treating, inhibiting, or preventing a condition or disorder characterized by a steroid hormone deficiency, and in particular, conditions or disorders characterized by low levels of estrogen and/or low levels of progesterone. The methods comprise administering to a subject a therapeutically effective amount of at least one pharmaceutical composition described herein, wherein the pharmaceutical composition comprises both estradiol and progesterone.

In particular, embodiments, the present disclosure provides a pharmaceutical composition suitable for administering a steroid hormone to a subject in need thereof, the pharmaceutical composition comprising estradiol, progesterone, a lipophilic surfactant system comprising a first lipophilic surfactant and a second lipophilic surfactant, wherein the first and second lipophilic surfactants are different from each other, a hydrophilic surfactant system comprising first and second hydrophilic surfactants, and an optional terpene, wherein the pharmaceutical composition is completely or substantially free of fractionated vegetable oils.

In certain embodiments, the first lipophilic surfactant is a first partial triglyceride.

In certain embodiments, the second lipophilic surfactant is a second partial triglyceride.

In certain embodiments, the first and second partial triglycerides are selected from the group consisting of IMWITOR 988, IMWITOR 742, IMWITOR 308, CAPMUL MCM NF, CAPMUL 708G, and glyceryl dilaurate.

In some embodiments, the first partial triglyceride is CAPMUL MCM NF and the second partial triglyceride is CAPMUL 708G.

In some embodiments, the first hydrophilic surfactant is a polyoxyethylene sorbitan fatty acid derivative.

In certain embodiments, the polyoxyethylene sorbitan fatty acid derivative is TWEEN 20 or TWEEN 80.

In certain embodiments, the second hydrophilic surfactant is a castor oil or hydrogenated castor oil ethoxylate.

In some embodiments, the castor oil or hydrogenated castor oil ethoxylate is CREMOPHOR EL, CREMOPHOR RH40, ETOCAS 40, CRODURET 60, or KOLLIPHOR HS 15.

In some embodiments, the castor oil or hydrogenated castor oil ethoxylate is CREMOPHOR RH40.

In some embodiments, the second hydrophilic surfactant is LABRASOL, TPGS, or ascorbyl-6 palmitate.

In some embodiments, the second hydrophilic surfactant is TPGS.

In certain embodiments, the terpene is not optional and is selected from the group consisting of d-limonene, menthene, menthol, phellandrene, terpinene, or terpineol.

In some embodiments, the terpene is d-limonene.

In some embodiments, the steroid hormone is a combination of estradiol and progesterone, or a combination of estradiol and a progesterone analog, or a combination of an estrogen analog and progesterone, or a combination of an estrogen analog and a progesterone analog.

The present disclosure further provides a method of treating a disease or condition associated with reduced estrogen levels and/or reduced progesterone levels, the method comprising administering to a subject in need thereof a pharmaceutical composition according to any of the preceding embodiments.

In some embodiments, the first lipophilic surfactant is a first partial triglyceride.

In some embodiments, the second lipophilic surfactant is a second partial triglyceride.

In certain embodiments, the first and second partial triglycerides are selected from the group consisting of IMWITOR 988, IMWITOR 742, IMWITOR 308, CAPMUL MCM NF, CAPMUL 708G, and glyceryl dilaurate.

In some embodiments, the first partial triglyceride is CAPMUL MCM NF and the second partial triglyceride is CAPMUL 708G.

In some embodiments, the first hydrophilic surfactant is a polyoxyethylene sorbitan fatty acid derivative.

In certain embodiments, the polyoxyethylene sorbitan fatty acid derivative is TWEEN 20 or TWEEN 80.

In certain embodiments, the second hydrophilic surfactant is a castor oil or hydrogenated castor oil ethoxylate.

In certain embodiments, the castor oil or hydrogenated castor oil ethoxylate is CREMOPHOR EL, CREMOPHOR RH40, ETOCAS 40, CRODURET 60, or KOLLIPHOR HS 15.

In some embodiments, the castor oil or hydrogenated castor oil ethoxylate is CREMOPHOR RH40.

In certain embodiments, the second hydrophilic surfactant is LABRASOL, TPGS, or ascorbyl-6 palmitate.

In certain embodiments, the second hydrophilic surfactant is TPGS.

In certain embodiments, the terpene is not optional and is selected from the group consisting of d-limonene, menthene, menthol, phellandrene, terpinene, or terpineol.

In some embodiments, the terpene is d-limonene.

In certain embodiments, the disease or condition associated with reduced estrogen levels and/or reduced progesterone levels is selected from the group consisting of symptoms associated with menopause, such as hot flushes/flashes, night sweats, vaginal dryness, urinary tract infections and loss of libido as well as other conditions and diseases such as heart disease and osteoporosis.

In certain embodiments, the disease or condition associated with reduced estrogen and/or progesterone levels is menopause. In certain embodiments, the disease or condition associated with reduced estrogen and/or progesterone levels is vasomotor symptoms of menopause, such as hot flushes/flashes, night sweats.

### BRIEF DESCRIPTION OF THE DRAWINGS/FIGURES

The foregoing summary, as well as the following detailed description, will be better understood when read in conjunction with the appended figures. For the purpose of illustration, the figures may describe the use of specific embodiments. It should be understood, however, that this disclosure is not limited to the precise embodiments discussed or described in these figures.

FIG. 1 is a graph of plasma concentration of progesterone vs. time for rats dosed with 20 μl of each of the various embodiments of the pharmaceutical composition described herein or 20 μl PROMETRIUM. Because of the way it is formulated, PROMETRIUM contains 400 mg progesterone/g of formulation. As such, the amount of progesterone dosed in rats treated with 20 μl PROMETRIUM far exceeded the amount of progesterone delivered to rats treated with 20 μl of the pharmaceutical compositions of this disclosure.

FIG. 2 is the log-linear version of FIG. 1.

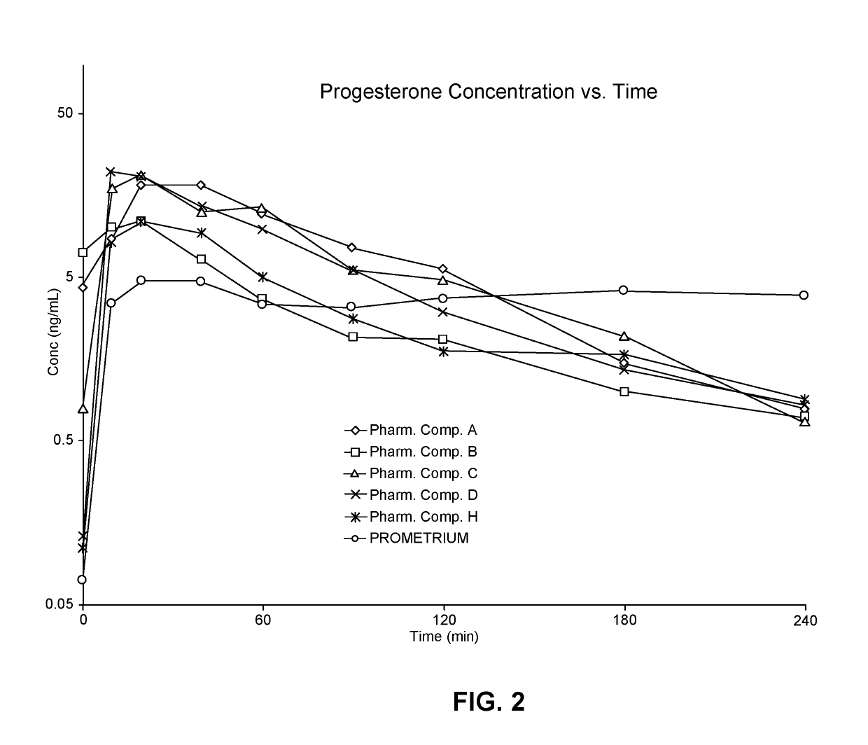

FIG. 3 is a graph of plasma concentration of progesterone metabolite allopregnanolone sulfate vs. time for various embodiments of the pharmaceutical composition described herein and PROMETRIUM. As discussed above, the amount of progesterone administered to rats treated with 20 μl PROMETRIUM far exceeded the amount of progesterone administered to rats treated with 20 μl of the pharmaceutical compositions of this disclosure.

FIG. 4 is a log-linear version of FIG. 3.

FIG. 5 is a graph of plasma concentration of progesterone metabolite 20α-dihydroprogesterone vs. time for various embodiments of the pharmaceutical composition described herein and PROMETRIUM. As discussed above, the amount of progesterone administered to rats treated with 20 μl PROMETRIUM far exceeded the amount of progesterone administered to rats treated with 20 μl of the pharmaceutical compositions of this disclosure.

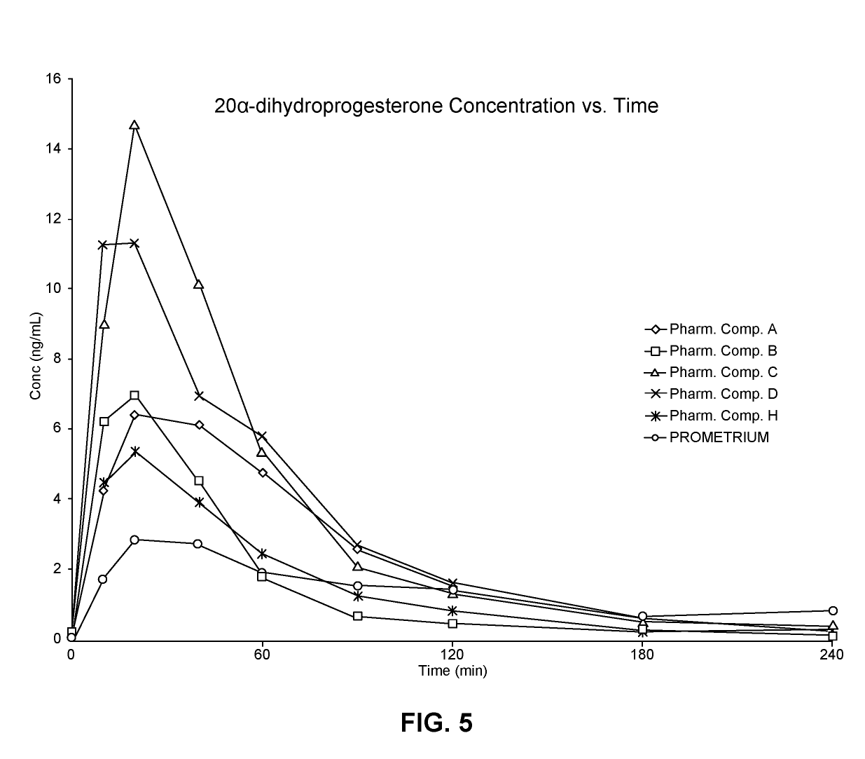

FIG. 6 is a log-linear version of FIG. 5.

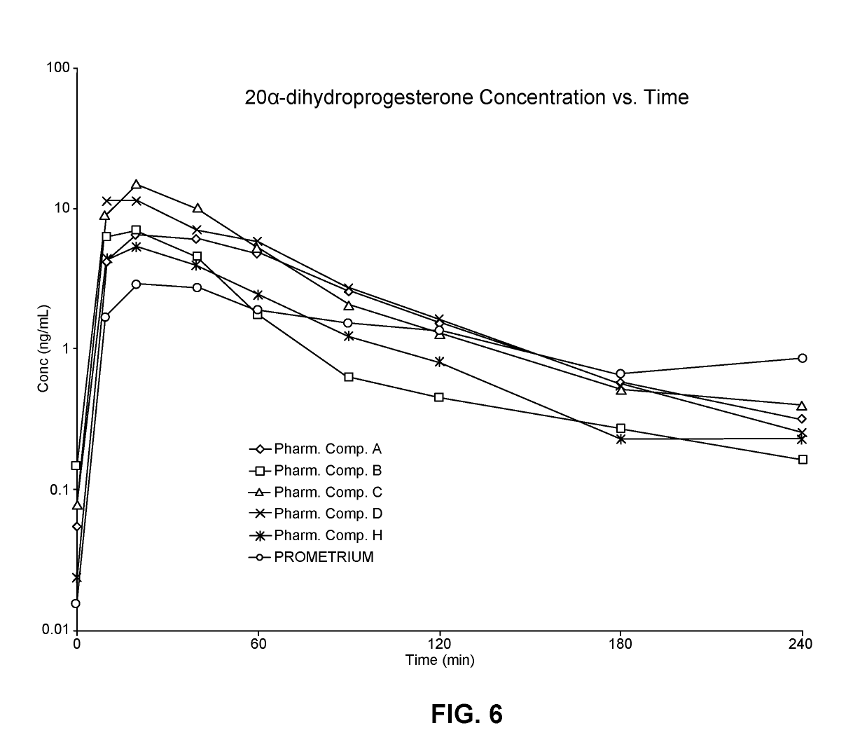

FIG. 7 is a graph of the plasma concentration of progesterone vs. time for fed and fasted rats dosed with 20 μl (25 mg/kg sample) of PROMETRIUM.

FIG. 8 is a graph of the plasma concentration of progesterone vs. time for fed and fasted rats dosed with 20 μl (3.7 mg/kg progesterone) of test pharmaceutical composition D in a gavage micro capsule.

FIG. 9 is a graph of the plasma concentration of progesterone vs. time for fed and fasted rats dosed with 20 μl (3.7 mg/kg progesterone) of test pharmaceutical composition C in a gavage micro capsule.

FIG. 10 is a graph of the plasma concentration of progesterone vs. time for fed and fasted rats dosed with 20 μl (3.7 mg/kg progesterone) of test pharmaceutical composition A in a gavage micro capsule.

FIG. 11 is graph of plasma concentration of progesterone vs. time for PROMETRIUM and test pharmaceutical composition D.

### DETAILED DESCRIPTION

### Definitions

The singular forms “a,” “an,” and “the” include plural referents unless the context clearly dictates otherwise.

As used herein, the term “or” is a logical disjunction (i.e., and/or) and does not indicate an exclusive disjunction unless expressly indicated as such with the terms “either,” “unless,” “alternatively,” and words of similar effect.

As used herein the term “bioidentical” means that a given compound, typically a hormone, is identical to or matches the chemical structure and effect of a compound that occurs naturally or endogenously in the human body.

As used herein, the term “about” refers to ±10% of the noted value, unless otherwise specified, and unless the upper bound of the range would exceed 100% of the pharmaceutical composition, in which case the upper limit of the range is limited to 99.9%. Thus, and by way of example only, a pharmaceutical composition including about 10 weight percent of a given compound could have from 9 to 11 weight percent of the compound. Similarly, a pharmaceutical composition including about 95 weight percent of a given compound could have from 85.5 to 99.9 weight percent of the compound in the pharmaceutical composition.

As used herein, the term “hormone deficiency” refers to a low level of one or more steroid hormones in a subject. Normal hormone levels will vary from subject to subject and can be determined via known methods. Low hormone levels may or may not be associated with symptoms including, but not limited to, fatigue, irregular bleeding, lowered libido, and depression. Conditions that can be treated with estrogen and progesterone therapy to address estrogen and progesterone deficiencies include menopause-related symptoms including vasomotor symptoms (e.g., hot flashes and night sweats). Other hypoestrogenism related conditions and symptoms can also be treated with estrogen and progesterone therapy, including, for example and without limitation, vasomotor symptoms, sleep disturbances, mood changes, and vulvo-vaginal atrophy; and osteoporosis and other non-menopausal disease states or conditions that can be treated with supplemental estradiol and progesterone.

As used herein, the terms “host,” “subject,” and “patient” refer to any animal, including humans.

The phrase “hydrophilic surfactant” refers to those surfactants having a hydrophilic-lipophilic balance (HLB) value greater than or equal to 10.

The phrase “lipophilic surfactant” refers to those surfactants having a hydrophilic-lipophilic balance (HLB) value less than 10.

The term “micronized” as used herein, refers to particles having an X50 particle size value below about 15 microns or having an X90 particle size value below about 25 microns. In some embodiments, a micronized particle can have an X90 particle size of less than 5 microns. The term “X50” means that one-half of the particles in a sample are smaller in diameter than a given number. For example, a micronized particle having an X50 of 5 microns means that, for a given sample of the micronized particle, one-half of the particles have a diameter of less than 5 microns. Similarly, the term “X90” means that ninety percent (90%) of the particles in a sample are smaller in diameter than a given number.

As used herein, the term “predominantly” means at least 50 percent. By way of example only, a compound comprising a linear predominantly C10 alkylene group, comprises at least 50 percent, at least 60 percent, at least 70 percent, at least 80 percent, at least 85 percent, at least 90 percent, at least 91 percent, at least 92 percent, at least 93 percent, at least 94 percent, at least 95 percent, at least 96 percent, at least 97 percent, at least 98 percent, or at least 99 percent of the linear C10 alkylene group, with the remainder being an alkylene group either greater than or less than C10. In certain embodiments, predominantly means at least 85 percent. “Predominately” can be used in a variety of unit measurement systems, including mol %, w/w, or aggregate number of fatty acid esters, for example.

As used herein, the term “prevent” refers to the prophylactic treatment of a subject who is at risk of developing a condition (e.g., steroid hormone deficiency) resulting in a decrease in the probability that the subject will develop the condition.

The term “estradiol” refers to (17β)-estra-1,3,5(10)-triene-3,17-diol. Estradiol is also interchangeably called 17β-estradiol, oestradiol, or E2, and is found endogenously in the human body. As used herein, estradiol refers to the bio-identical or body-identical form of estradiol found in the human body having the structure:

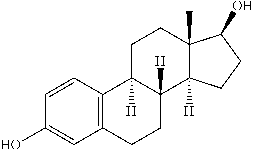

Estradiol is supplied in an anhydrous or hemi-hydrate form. For the purposes of this disclosure, the anhydrous form or the hemihydrate form can be substituted for the other by accounting for the water or lack of water according to well-known and understood techniques.

The term “solubilized estradiol” means that the estradiol or a portion thereof is solubilized or dissolved in the solubilizing agent(s) or the formulations disclosed herein. Solubilized estradiol may include estradiol that is about 80% solubilized, about 85% solubilized, about 90% solubilized, about 95% solubilized, about 96% solubilized, about 97% solubilized, about 98% solubilized, about 99% solubilized or about 100% solubilized. In some embodiments, the estradiol is “fully solubilized” with all or substantially all of the estradiol being solubilized or dissolved in the solubilizing agent. Fully solubilized estradiol may include estradiol that is about 97% solubilized, about 98% solubilized, about 99% solubilized or about 100% solubilized. Solubility can be expressed as a mass fraction (% w/w, which is also referred to as weight percent (wt %)).

As used herein, the term “progesterone” refers to pregn-4-ene-3,20-dione. As used herein, progesterone refers to the bioidentical or body-identical form of progesterone found in the human body having the structure:

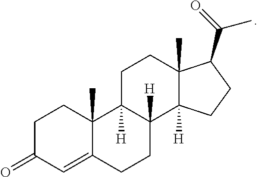

The term “solubilized progesterone” means that the progesterone or a portion thereof is solubilized or dissolved in the formulations disclosed herein. Solubilized progesterone may include progesterone that is about 80% solubilized, about 85% solubilized, about 90% solubilized, about 95% solubilized, about 96% solubilized, about 97% solubilized, about 98% solubilized, about 99% solubilized or about 100% solubilized. In some embodiments, the progesterone is “fully solubilized” with all or substantially all of the progesterone being solubilized or dissolved in the formulation. Fully solubilized progesterone may include progesterone that is about 97% solubilized, about 98% solubilized, about 99% solubilized or about 100% solubilized. Solubility can be expressed as a mass fraction (% w/w, which is also referred to as weight percent (wt %)).

The solubility of a given steroid hormone can be measured using standard techniques by weighing a piece of filter paper, placing the weighed filter paper in a buchner funnel (porcelain or glass with a glass frit), and drawing a known quantity of pharmaceutical composition through the filter paper using vacuum (such as with a side-arm flask fitted with a neoprene collar). After drying for an appropriate period of time (either at room temperature or at elevated temperature), the filter paper is reweighed. The amount of steroid hormone on the filter paper is calculated and the amount of solubilized and insoluble steroid hormone is calculated.

The terms “treat,” “treating,” “treatment” and the like refer to any indicia of success in the treatment or amelioration of an injury, disease, or condition, including any objective or subjective parameter such as abatement; remission; diminishing of symptoms or making the injury, disease, or condition more tolerable to the patient; slowing in the rate of degeneration or decline; or improving a patient's physical or mental well-being. The treatment or amelioration of symptoms can be based on objective or subjective parameters, including the results of a physical examination, neuropsychiatric examinations, or psychiatric evaluation.

The phrase “therapeutically effective amount” refers to an amount of a pharmaceutical composition or of a given steroid hormone suitable to treat a particular disorder or disease.

As used herein, the phrase “substantially” means at least about 90%, in certain embodiments, at least about 95%, and in still further embodiments, at least about 98%. For example, an object that is “substantially pure” or an object that is “substantially free” of another object, refers to a compound or composition that is at least about 90% pure by weight, at least about 95% pure by weight, or at least about 98% pure by weight and contains less than about 10% by weight, less than about 5% by weight or less than about 2% by weight of contaminants.

As used herein, the phrase “steroid hormone” refers to estradiol, 17β-estradiol, oestradiol, E2, progesterone, 17-hydroxyprogesterone, or 5α-dihydroprogesterone.

As used herein, the term “d-limonene” refers to (4R)-1-methyl-4-(1-methylethenyl)-cyclohexene (CAS No. 5989-27-5), which is also known by synonyms including (+)-4-isopropenyl-1-methylcyclohexene, (+)-p-mentha-1,8-diene, and (R)-(+)-Limonene.

The term “area under the curve” (“AUC”) refers to the area under the curve defined by changes in the blood concentration of an active pharmaceutical ingredient (e.g., estradiol or progesterone), or a metabolite of the active pharmaceutical ingredient, over time following the administration of a dose of the active pharmaceutical ingredient. “AUC_{0-∞}” is the area under the concentration-time curve extrapolated to infinity following the administration of a dose. “AUC₀₋ₜ” is the area under the concentration-time curve from time zero to time t following the administration of a dose, wherein t is the last time point with a measurable concentration.

The term “Cₘₐₓ” refers to the maximum value of blood concentration shown on the curve that represents changes in blood concentrations of an active pharmaceutical ingredient (e.g., progesterone or estradiol), or a metabolite of the active pharmaceutical ingredient, over time.

The term “Tₘₐₓ” refers to the time that it takes for the blood concentration of an active pharmaceutical ingredient (e.g., estradiol or progesterone), or a metabolite of the active pharmaceutical ingredient, to reach its maximum value.

The term “bioavailability,” which has the meaning defined in 21 C.F.R. §320.1(a), refers to the rate and extent to which an active ingredient or active moiety is absorbed from a drug product and becomes available at the site of action. For drug products that are not intended to be absorbed into the bloodstream, bioavailability may be assessed by measurements intended to reflect the rate and extent to which the active ingredient or active moiety becomes available at the site of action. For example, bioavailability can be measured as the amount of active ingredient in the blood (serum or plasma) as a function of time. Pharmacokinetic (PK) parameters such as AUC, Cₘₐₓ, or tₘₐₓ may be used to measure and assess bioavailability.

The term “bioequivalent,” has the meaning defined in 21 C.F.R. §320.1(e) and refers to the absence of a significant difference in the rate and extent to which the active ingredient or active moiety in pharmaceutical equivalents or pharmaceutical alternatives becomes available at the site of drug action when administered at the same molar dose under similar conditions in an appropriately designed study. Where there is an intentional difference in rate (e.g., in certain extended release dosage forms), certain pharmaceutical equivalents or alternatives may be considered bioequivalent if there is no significant difference in the extent to which the active ingredient or moiety from each product becomes available at the site of drug action. This applies only if the difference in the rate at which the active ingredient or moiety becomes available at the site of drug action is intentional and is reflected in the proposed labeling, is not essential to the attainment of effective body drug concentrations on chronic use, and is considered medically insignificant for the drug. In practice, two products are considered bioequivalent if the 90% confidence interval of the AUC or Cₘₐₓ is within 80.00% to 125.00%.

The term “bio-identical hormone” refers to an active pharmaceutical ingredient that is structurally identical to a hormone naturally or endogenously found in the human body (e.g., estradiol and progesterone).

The term “polyoxyethylene sorbitan fatty acid derivative” refers to a compound having the structure:

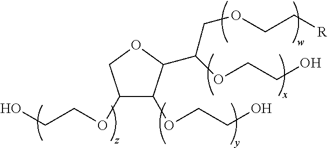

wherein w+x+y+z ranges from about 10 to about 50, and in particular embodiments, from about 10 to about 30, and wherein R is a C₆-C₁₈ fatty acid radical. Exemplary polysorbates within the scope of the present definition include, but are not limited to, polysorbate 20, polysorbate 40, polysorbate 60, polysorbate 65, and polysorbate 80.

### Pharmaceutical Compositions

The pharmaceutical compositions disclosed herein are capable of fully solubilizing steroid hormones, and in particular, estradiol and progesterone. Surprisingly, the pharmaceutical compositions in this disclosure provide a significantly better pharmacokinetic (“PK”) profile for steroid hormones, and estradiol and progesterone in particular, in a subject in need thereof than currently marketed pharmaceutical compositions, such as ESTRACE and PROMETRIUM. For instance, pharmaceutical compositions comprising progesterone achieve this enhanced PK profile despite containing from about ⅙ to about ⅛ as much progesterone as a comparable volume of PROMETRIUM. PROMETRIUM, for example, contains approximately 400 mg of progesterone per gram of formulation, while the pharmaceutical compositions provided in this disclosure contain, in certain embodiments, from about 10 to about 100 mg progesterone per gram of pharmaceutical composition, and in certain embodiments, about 60 mg progesterone per gram of pharmaceutical composition. Thus, and by way of example only, if a human subject were administered a 500 mg gel cap (a common gelcap size) of PROMETRIUM or a gelcap containing 500 mg of a pharmaceutical compositions disclosed herein comprising about 6 weight percent progesterone, the PROMETRIUM dose would contain 200 mg of progesterone compared to only 30 mg of progesterone in the exemplary pharmaceutical composition of this disclosure. Thus, the human receiving the exemplary composition would receive significantly less progesterone than the subject dosed with PROMETRIUM. Despite the significant difference in the amount of progesterone dosed, it has now been surprisingly found, that the present compositions provide significantly increased bioavailability compared to PROMETRIUM. The enhanced bioavailability of progesterone and/or estradiol in the present composition allows for a significant reduction in the amount progesterone and/or estradiol that must be administered to a subject per dose to achieve the same or better results as PROMETRIUM and/or ESTRACE.

Without wishing to be bound by any particular theory, it is believed that, in certain embodiments, the described pharmaceutical compositions form micelles upon administration that both protect the estradiol and progesterone from the digestive milieu and facilitate absorption of the estradiol and progesterone across the gut mucosa and into the blood stream. That said, in other embodiments, and without wishing to be bound by any particular theory, the enhanced bioavailability observed in all of the present compositions may be due to the fully-solubilized nature of the estradiol and the progesterone present in the compositions and the absence of suspended (insoluble) estradiol and progesterone. Thus, in some embodiments, the pharmaceutical composition can be characterized as a fully-solubilized estradiol and progesterone pharmaceutical composition capable of forming micelles. Other embodiments, however, may comprise fully-solubilized estradiol and fully-solubilized progesterone, but may not form micelles. In still other embodiments, the presence of both fully-solubilized estradiol and fully solubilized progesterone and the formation of micelles together in the same pharmaceutical composition may result in an effect that further enhances the bioavailability of the estradiol and progesterone above the bioavailability that would result if either only micelles were formed, or only fully-solubilized estradiol and fully-solubilized progesterone were present.

Micelle formation can be observed by adding the pharmaceutical compositions as described herein to water or other aqueous-based fluid such as simulated gastric fluid (SGF). The size or size distribution of the micelles resulting from mixing the present pharmaceutical compositions with water or SGF can be measured using a photon correlation spectroscopy. In certain embodiments, the particles can have a size distribution ranging from about 1 nm to about 1400 nm in water, or from about 130 nm to about 465 nm in water, or from about 100 nm to about 210 nm in water.

In certain embodiments, the micelles can have a zeta potential (mV) ranging from about −10 to about −30 mV. In certain embodiments, the zeta potential of the micelles can be about −10 mV, about −11 mV, about −12 mV, about −13 mV, about −14 mV, about −15 mV, about −16 mV, about −17 mV, about −18 mV, about −19 mV, about −20 mV, about −21 mV, about −22 mV, about −23 mV, about −24 mV, about −25 mV, about −26 mV, about −27 mV, about −28 mV, about −29 mV, or about −30 mV. In certain embodiments, the zeta potential can be about −16 to about −17 mV. In other embodiments, the zeta potential can be about −18 to about −19 mV. In still other embodiments, the zeta potential can be about −20 to about −21 mV.

In certain embodiments, this disclosure provides pharmaceutical compositions capable of forming micelles, the compositions comprising estradiol, progesterone, at least one lipophilic surfactant, at least one hydrophilic surfactant, and, optionally, a terpene.

In some embodiments, the pharmaceutical composition capable of forming micelles comprises estradiol, progesterone, a lipophilic surfactant system, a hydrophilic surfactant system, and, optionally, a terpene.

In still further embodiments, the pharmaceutical composition capable of forming micelles comprises estradiol, progesterone, a lipophilic surfactant system comprising a first lipophilic surfactant and a second lipophilic surfactant, a hydrophilic surfactant system comprising a first hydrophilic surfactant and a second hydrophilic surfactant, and, optionally, a terpene.

In yet another embodiment, the present disclosure provides non-micelle forming pharmaceutical compositions comprising estradiol, progesterone, and a lipophilic surfactant in the complete or substantial absence of a hydrophilic surfactant.

In another embodiment, the present disclosure provides non-micelle forming pharmaceutical compositions comprising a steroid hormone and a lipophilic surfactant system in the complete or substantial absence of hydrophilic surfactants.

In still another embodiment, the present disclosure provides non-micelle forming pharmaceutical compositions comprising a steroid hormone and a lipophilic surfactant system comprising a first lipophilic surfactant and a second lipophilic surfactant, all in the complete or substantial absence of hydrophilic surfactants

In all of the pharmaceutical compositions described herein, the steroid hormones are estradiol and progesterone.

### Lipophilic Surfactants

Lipophilic surfactants suitable for use in the pharmaceutical compositions disclosed herein are those lipophilic surfactants having an HLB value less than 10. Exemplary lipophilic surfactants having the desired HLB value include, but are not limited to fatty acids and esters thereof (e.g., C₆-C₁₄ fatty acids, C₇-C₁₂ fatty acids, C₈-C₁₀ fatty acids, or C₈ fatty acids, or C₁₀ fatty acids). Exemplary fatty acids include, but are not limited to, caprylic acid, capric acid, octanoic acid, decanoic acid, undecanoic acid, lauric acid, and myristic acid. In some embodiments, the fatty acids are saturated. In other embodiments, the fatty acids contain at least one double bond, and in certain embodiments, 2, 3, or 4 double bonds.

Other suitable lipophilic surfactants can be partial triglycerides. Partial triglycerides are fatty acid mono-esters of glycerol, fatty acid di-esters of glycerol, and, in certain embodiments, combinations of these mono- and diglycerides. Diglycerides can be esterified with the same or different fatty acids. Partial triglycerides are well known in the art and are widely commercially available.

Because of the way in which partial triglycerides are produced, they often contain small amounts of impurities. These impurities include, for example, di- and triglycerides in the case of monoglycerides and mono- and tri-glycerides in the case of diglycerides. Additionally, because many fatty acids are naturally sourced, they often contain, in addition to fatty acids having the desired chain length, fatty acids having either longer or shorter chain lengths than the preferred fatty acid(s). Because these impurities are present in small amounts and are difficult to remove, they are carried through into the esterification processes used to prepare the partial triglycerides. As a result, small quantities of mono-, di-, and triglycerides esterified with fatty acids having a chain length other than the desired chain length can be present in any given partial triglyceride composition. However, because these undesired mono-, di-, and triglycerides are present at sufficiently low amounts, their presence does not affect or contribute to the efficacy or utility of the partial triglyceride(s) making up the vast majority of a given commercially available product.

For purposes of this disclosure, “partial triglycerides” are compositions comprising one or more compounds according to Formula I:

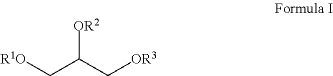

wherein R¹, R², and R³ are each independently H or a C₆-C₁₄ fatty acid radical having the structure —C(═O)R⁴, wherein each R⁴ is, independently at each occurrence, a linear predominantly C5 alkylene group, a linear predominantly C6 alkylene group, a linear predominantly C7 alkylene group, a linear predominantly C8 alkylene group, a linear predominantly C9 alkylene group, a linear predominantly C10 alkylene group, a linear predominantly C11 alkylene group, a linear predominantly C12 alkylene group, or a linear predominantly C13 alkylene group, each alkylene group optionally including one or more double bonds and each alkylene group optionally substituted at least once with —OH or —NH₂; with the proviso that the composition can include impurities wherein R¹, R², and R³ are all other than H at less than about 20 weight percent, less than about 15 weight percent, less than about 10 weight percent, less than about 9 weight percent, less than about 8 weight percent, less than about 7 weight percent, less than about 6 weight percent, less than about 5 weight percent, less than about 4 weight percent, less than about 3 weight percent, less than about 2 weight percent, or less than about 1 weight percent and impurities wherein all three of R¹, R², and R³ are H (i.e., glycerol) at less than about 5 weight percent, less than about 3 weight percent, less than about 1 weight percent, less than about 0.1 weight percent, less than about 0.01 weight percent, or wherein glycerol is completely absent. In certain embodiments, compounds wherein R¹, R², and R³ are all other than H are present with the desired compound(s) at less than about 10 weight percent. In other embodiments, compounds wherein R¹, R², and R³ are all other than H are present with the desired compound(s) at less than about 5 weight percent.

In some embodiments, the partial triglyceride can be a mixture of partial triglycerides. In one such embodiment, the mixture can be a mixture of partial triglycerides wherein each R⁴ can be, independently, a linear predominantly C7 alkylene or a linear predominantly C9 alkylene, with the proviso that impurities wherein R¹, R², and R³ are all other than H comprise less than about 20, less than about 15, less than about 10, less than about 9, less than about 8, less than about 7, less than about 6, less than about 5, less than about 4, less than about 3, less than about 2, or less than about 1 weight percent of the mixture. In certain embodiments of this mixture, about 60% of the mixture can be monoglycerides wherein R⁴ is a linear predominantly C7 alkylene or a linear predominantly C9 alkylene, while about 35% of the mixture can be diglycerides wherein each R⁴ can be, independently, a predominantly C7 or predominantly C9 alkylene group. In this embodiment, the weight ratio of predominantly C7 to predominantly C9 groups can range from about 75 to about 25 to about 85 to about 15. In particular embodiments, the weight ratio of predominantly C7 to predominantly C9 groups can be about 83 to about 17. Commercially available examples of such a mixture of partial triglycerides are CAPMUL MCM NF and CAPMUL MCM EP.

In other embodiments, the partial triglyceride can be a monoglyceride wherein each R⁴ can be a linear predominantly C7 alkylene group, with the proviso that compounds wherein at least two of R¹, R², and R³ are other than H comprise less than about 20, less than about 15, less than about 10, less than about 9, less than about 8, less than about 7, less than about 6, less than about 5, less than about 4, less than about 3, less than about 2, or less than about 1 weight percent of the partial triglyceride. An example of a partial triglyceride (monoglyceride) having the noted components and purity is glyceryl monocaprylate, commercially available as CAPMUL 708G.

Various commercially available partial triglycerides having an HLB value of less than 10 and falling within the scope of the definition provided above are known to those of ordinary skill in the art and include, but are not limited to, IMWITOR 988 (glyceryl mono-/di-caprylate, available from Sasol), IMWITOR 742 (caprylic/capric glycerides, available from Sasol), IMWITOR 308 (glyceryl mono-caprylate, available from Cremer Oleo Division), CAPMUL MCM NF (glyceryl caprylate/caprate, available from Abitec Corp.), CAPMUL 708G (glyceryl monocaprylate, available from Abitec Corp.), and glyceryl dilaurate.

Other suitable lipophilic surfactants having an HLB value of less than 10 are triglycerides. Suitable triglycerides include those triglycerides prepared from the esterification of glycerol with one or more predominantly medium chain (i.e. C₆-C₁₄) fatty acid optionally including one or more double bonds and optionally substituted at least once with —OH or —NH₂. Suitable triglycerides known to those of skill in the art include, but are not limited to MIGLYOL 808 (tricaprylin), MIGLYOL 810 (caprylic/capric triglyceride), and MIGLYOL 8108 (caprylic/capric triglyceride), each of which is available from Sasol.

In other embodiments, the lipophilic surfactant having an HLB value less than 10 can be a glycol fatty acid ester. In certain embodiments, the glycol is ethylene glycol, propylene glycol, polyethylene glycol, or polypropylene glycol, or a combination of any of these. Glycol fatty acid esters are well known in the art and can be obtained by esterifying a glycol, or combination of glycols, with one or more predominantly medium chain fatty acids as described above.

Exemplary propylene glycol mono- and di-esters of fatty acids of the type noted above include, but are not limited to LAUROGLYCOL 90 (propylene glycol monolaurate, available from Gattefosse), propylene glycol monomyristate, CAPTEX 200 (propylene glycol dicaprylate/dicaprate, available from Abitec Corp.), MIGLYOL 840 (propylene glycol dicaprylocaprate (dicaprylate/dicaprate), available from Sasol and Cremer Oleo GmbH & Co.) and NEOBEE M-20 (propylene glycol di (Caprylate/Caprate), available from Stepan). An exemplary polyethylene glycol diester is LIPOPEG 2-DL (PEG-4 dilaurate, available from Vantage Specialty Ingredients).

Further suitable lipophilic surfactants include acetic, succinic, lactic, citric or tartaric esters of mono- or di-glycerides of fatty acids, for example, MYVACET 9-45 (distilled acetylated monoglycerides, available from Sheffield Bioscience), Miglyol 829 (caprylic/capric diglyceryl succinate, available from Cremer Oleo Division), mono/di-succinylated monoglycerides, IMWITOR 372 P (glyceryl stearate citrate, available from Sasol), and IMWITOR 375 (Glyceryl Citrate/Lactate/Linoleate/Oleate, available from Sasol).

Further suitable lipophilic surfactants having the desired HLB value include polyglycerol esters of fatty acids such as PLUROL Oleique CC 497 (polyglyceryl-3 oleate, available from Gattefosse), CAPROL ET (polyglyceryl-6 octastearate, available from Abitec), and DREWPOL 10-10-O (decaglyceryl decaoleate, available from Stepan). Castor oil ethoxylates of low ethoxylate content (HLB<10) such as ETOCAS 5 (polyoxyethylene (5) castor oil, available from Croda) can also be used.

Other lipophilic surfactants having an HLB value less than 10 include fatty acid sorbitan esters, for example, SPAN 20 (sorbitan monolaurate, available from SIGMA-ALDRICH), and SPAN 80 (sorbitan oleate, available from Croda).

Transesterification products of natural or hydrogenated vegetable oil triglyceride and a polyalkylene polyol can also be used as the lipophilic surfactant having an HLB value less than 10. Examples include, but are not limited to, LABRAFIL M1944CS (oleoyl polyoxyl-6-glycerides NF, available from Gattefosse), and LABRAFIL M2125CS (linoleoyl macrogol-6-glycerides EP, available from Gattefosse).

Other suitable lipophilic surfactants having an HLB value less than 10 include alcohol ethyoxylates, e.g. BRIJ O3 (Oleth-3, available from Croda), BRIJ O2 (Oleth-2, available from Croda), BRIJ L4 (Laureth-4, available from Croda), and PLURONICS, for example, polyoxyethylene-polyoxypropylene co-polymers and block co-polymers e.g. SYNPERONIC PE/L42 and SYNPERONIC PE/L62, both available from Croda.

### The Lipophilic Surfactant System

As discussed previously, in certain embodiments, this disclosure provides pharmaceutical compositions comprising estradiol, progesterone, at least one lipophilic surfactant, at least one hydrophilic surfactant, and, optionally, a terpene. In certain embodiments, the at least one lipophilic surfactant can be any of the lipophilic surfactants discussed above.

In other embodiments, however, the at least one lipophilic surfactant can be a lipophilic surfactant system. In certain embodiments, the lipophilic surfactant system can comprise a first lipophilic surfactant and a second lipophilic surfactant different from the first. In other embodiments, the lipophilic surfactant system can comprise a first lipophilic surfactant, a second lipophilic surfactant, and a third lipophilic surfactant, wherein each of the first, second, and third lipophilic surfactants are different from each other. In still further embodiments, the lipophilic surfactant system can comprise a first lipophilic surfactant, a second lipophilic surfactant, a third lipophilic surfactant, and a fourth lipophilic surfactant wherein each of the first, second, third, and fourth lipophilic surfactants are different from each other. In still further embodiments, the lipophilic surfactant system can comprise a first lipophilic surfactant, a second lipophilic surfactant, a third lipophilic surfactant, a fourth lipophilic surfactant, and a fifth lipophilic surfactant, wherein each of the first, second, third, fourth, and fifth lipophilic surfactants are different from each other. For each of these embodiments, the first, second, third, fourth, and fifth lipophilic surfactants can be selected from any of the suitable lipophilic surfactants discussed above.

In embodiments where the lipophilic surfactant system comprises first and second lipophilic surfactants, wherein the first and second lipophilic surfactants are different from each other, the first lipophilic surfactant can comprise from about 1 weight percent to about 99 weight percent of the lipophilic surfactant system, with the remainder comprising the second lipophilic surfactant. In particular embodiments, the first lipophilic surfactant can comprise from about 10 weight percent to about 99 weight percent of the lipophilic surfactant system, from about 20 weight percent to about 99 weight percent of the lipophilic surfactant system, from about 30 weight percent to about 99 weight percent of the lipophilic surfactant system, from about 40 weight percent to about 99 weight percent of the lipophilic surfactant system, from about 50 weight percent to about 99 weight percent of the lipophilic surfactant system, from about 60 weight percent to about 99 weight percent of the lipophilic surfactant system, from about 70 weight percent to about 99 weight percent of the lipophilic surfactant system, from about 80 weight percent to about 99 weight percent of the lipophilic surfactant system, from about 90 weight percent to about 99 weight percent of the lipophilic surfactant system, from about 90 weight percent to about 95 weight percent of the lipophilic surfactant system. In certain embodiments, the first lipophilic surfactant can comprise from about 85 weight percent to about 95 weight percent of the lipophilic surfactant system, or from about 88 to about 92 weight percent of the lipophilic surfactant system.

In certain embodiments, the lipophilic surfactant system comprises about 90 weight percent of the first lipophilic surfactant and about 10 weight percent of the second lipophilic surfactant. In an alternative embodiment, the lipophilic surfactant system comprises 90 weight percent of the first lipophilic surfactant and 10 weight percent of the second lipophilic surfactant.

In certain embodiments, the lipophilic surfactant system comprises about 95 weight percent of the first lipophilic surfactant and about 5 weight percent of the second lipophilic surfactant. In an alternative embodiment, the lipophilic surfactant system comprises 95 weight percent of the first lipophilic surfactant and 5 weight percent of the second lipophilic surfactant.

The lipophilic surfactant system can comprise from about 30 weight percent to about 95 weight percent of the pharmaceutical composition. In particular embodiments, the lipophilic surfactant system can comprise from about 40 weight percent to about 95 weight percent of the pharmaceutical composition, from about 50 weight percent to about 95 weight percent of the pharmaceutical composition, from about 60 weight percent to about 95 weight percent of the pharmaceutical composition, from about 70 weight percent to about 95 weight percent of the pharmaceutical composition, from about 75 weight percent to about 95 weight percent of the pharmaceutical composition, from about 75 weight percent to about 85 weight percent of the pharmaceutical composition, or about 80 weight percent of the pharmaceutical composition.

In some embodiments, the first and second lipophilic surfactants can be first and second partial triglycerides, respectively, wherein the first partial triglyceride is different from the second partial triglyceride.

In embodiments comprising a first and second partial triglyceride, the first and second partial triglycerides can be independently selected from the group consisting of IMWITOR 988, IMWITOR 742, IMWITOR 308, CAPMUL MCM NF, CAPMUL 708G, with the proviso that the first and second partial triglycerides are different.

In some embodiments, the first and second lipophilic surfactants can be CAPMUL MCM NF and CAPMUL 708G. In certain embodiments, the CAPMUL 708G can be about 90 or about 95 weight percent of the lipophilic surfactant system, with CAPMUL MCM NF, comprising the remaining amount of the surfactant system.

### Hydrophilic Surfactants

Hydrophilic surfactants suitable for use in the pharmaceutical compositions disclosed herein include those hydrophilic surfactants known to those of ordinary skill in the art and having an HLB value greater than or equal to 10. Examples include, but are not limited to polyoxyethylene sorbitan fatty acid derivatives, e.g., TWEEN 20 (polyethylene glycol sorbitan monolaurate; polysorbate 20; available from Sigma-Aldrich), TWEEN 80 (polyethylene glycol sorbitan monooleate; polysorbate 80; available from Sigma-Aldrich), and MONTANOX 40 (polyethylene glycol sorbitan monopalmitate; polysorbate 40; available from Sigma-Aldrich).

Other suitable hydrophilic surfactants having the desired HLB value include castor oil or hydrogenated castor oil ethoxylates, e.g., CREMOPHOR EL (polyoxyl 35 castor oil USP, available from BASF), CREMOPHOR RH40 (KOLLIPHOR RH 40; polyoxyl 40 hydrogenated castor oil USP, available from BASF), ETOCAS 40 (PEG 40 castor oil, available from Croda), CRODURET 60 (PEG-60 hydrogenated castor oil, available from Croda), and KOLLIPHOR HS 15 (polyethylene glycol 15-hydroxystearate, available from Sigma-Aldrich).

Other suitable hydrophilic surfactants include LABRASOL (caprylocaproyl macrogol-8 glycerides EP, available from Gattefosse), ascorbyl palmitate (available from Sigma-Aldrich), and d-α-tocopherol polyethylene glycol succinate derivatives having the formula:

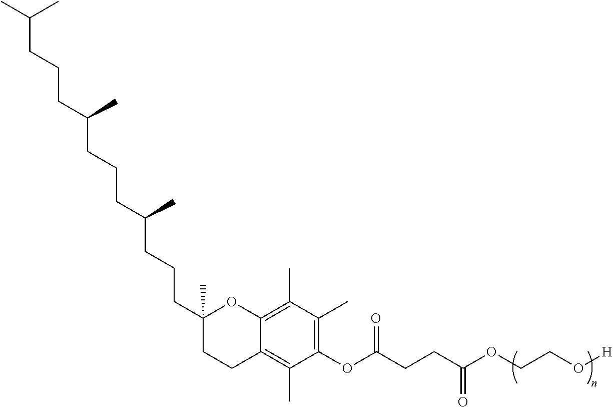

wherein n can range from 1 to about 100, and in particular embodiments, from about 1 to about 50 or about 1 to about 25. In particular embodiments, the d-α-tocopherol polyethylene glycol succinate derivative can be d-α-tocopherol polyethylene glycol 1000 succinate, also referred to as TPGS-1000 and TPGS (n≈22). TPGS-1000 is available from Sigma-Aldrich.

Further suitable hydrophilic surfactants having the desired HLB value include the GELUCIREs, including GELUCIRE 50/13 (Stearoyl macrogol-32 glycerides EP/Stearoyl polyoxyl-32 glycerides NF, available from Gattefosse); fatty acid ethoxylates, e.g., MYRJ S8 (polyoxyethylene (8) stearate, available from Croda), PEG-30 glyceryl laurate (available from MakingCosmetics, Snoqualmie, Wash.), and PEG-20 glyceryl stearate; alcohol ethoxylates such as BRIJ O10 (polyoxyethylene (10) oleyl ether; Oleth-10; available from Croda); polyoxyethylene-polyoxypropylene co-polymers and block co-polymers, such as PLURONIC F-68 (Poloxamer 188, available from Sigma-Aldrich) and Poloxamer 407 (available from Sigma-Aldrich); and anionic surfactants such as sodium lauryl sulphate, sodium oleate, and sodium dioctylsulphosuccinate.

### Hydrophilic Surfactant Systems

As discussed previously, in certain embodiments, this disclosure provides pharmaceutical compositions comprising estradiol, progesterone, at least one lipophilic surfactant, at least one hydrophilic surfactant, and, optionally, a terpene. In certain embodiments, the at least one hydrophilic surfactant can be any of the hydrophilic surfactants discussed above.

In other embodiments, however, the at least one hydrophilic surfactant can be a hydrophilic surfactant system. In certain embodiments, the hydrophilic surfactant system can comprise a first hydrophilic surfactant and a second hydrophilic surfactant. The first and second hydrophilic surfactants can be selected from any of the suitable hydrophilic surfactants discussed above.

In certain embodiments, the first hydrophilic surfactant can comprise from about 1 weight percent to about 99 weight percent of the hydrophilic surfactant system, with the remainder comprising the second hydrophilic surfactant. In particular embodiments, the first hydrophilic surfactant can comprise from about 10 weight percent to about 99 weight percent of the hydrophilic surfactant system, from about 20 weight percent to about 99 weight percent of the hydrophilic surfactant system, from about 30 weight percent to about 99 weight percent of the hydrophilic surfactant system, from about 40 weight percent to about 99 weight percent of the hydrophilic surfactant system, from about 50 weight percent to about 99 weight percent of the hydrophilic surfactant system, from about 60 weight percent to about 99 weight percent of the hydrophilic surfactant system, from about 70 weight percent to about 99 weight percent of the hydrophilic surfactant system, from about 80 weight percent to about 99 weight percent of the hydrophilic surfactant system, from about 90 weight percent to about 99 weight percent of the hydrophilic surfactant system, from about 90 weight percent to about 95 weight percent of the hydrophilic surfactant system.

In particular embodiments, the first and second hydrophilic surfactants can each comprise about 50 weight percent of the hydrophilic surfactant system. In other embodiments, the first hydrophilic surfactant can comprise about 75 weight percent of the hydrophilic surfactant system, with the second hydrophilic surfactant comprising the remainder of the hydrophilic surfactant system.

The hydrophilic surfactant system can comprise from about 5 weight percent to about 15 weight percent of the pharmaceutical composition. In particular embodiments, the hydrophilic surfactant system can comprise from about 7 weight percent to about 12 weight percent of the pharmaceutical composition, from about 8 weight percent to about 11 weight percent of the pharmaceutical composition, from about 8 weight percent to about 10 weight percent of the pharmaceutical composition, from about 9 weight percent to about 10 weight percent of the pharmaceutical composition, from about 9.2 weight percent to about 9.6 weight percent of the pharmaceutical composition, from about 9.3 weight percent to about 9.5 weight percent of the pharmaceutical composition, or about 9.4 weight percent of the pharmaceutical composition.

In certain embodiments, the first hydrophilic surfactant can be a polyoxyethylene sorbitan fatty acid derivative. In further embodiments, the polyoxyethylene sorbitan fatty acid derivative can be TWEEN 20 (polysorbate 20) or TWEEN 80 (polysorbate 80). In still further embodiments, the first hydrophilic surfactant can be TWEEN 80.

In certain embodiments, the second hydrophilic surfactant can be a castor oil or hydrogenated castor oil ethoxylate. In particular embodiments, the castor oil or hydrogenated castor oil ethoxylate can be CREMOPHOR EL, CREMOPHOR RH40, ETOCAS 40, CRODURET 60, or KOLLIPHOR HS 15. In particular embodiments, the second hydrophilic surfactant can be KOLLIPHOR RH 40.

In other embodiments, the second hydrophilic surfactant can be LABRASOL, TPGS 1000, or ascorbyl-6 palmitate. In particular embodiments, the second hydrophilic surfactant can be TPGS 1000.

In particular embodiments, the first hydrophilic surfactant can be TWEEN 80. In certain embodiments, the TWEEN 80 can comprise about 50 weight percent of the hydrophilic surfactant system. In other embodiments, the TWEEN 80 can comprise about 75 weight percent of the hydrophilic surfactant system.

In certain embodiments, the second hydrophilic surfactant can be TPGS 1000 or PHOR RH 40. In certain embodiments, either the TPGS 1000 or the KOLLIPHOR RH 40 can be about 50 weight percent of the hydrophilic surfactant system. In other embodiments, either the TPGS 1000 or the KOLLIPHOR RH 40 can be about 25 weight percent of the hydrophilic surfactant system.

In certain embodiments, the first hydrophilic surfactant can be TWEEN 80 and the second hydrophilic surfactant can be TPGS 1000. In other embodiments, the first hydrophilic surfactant can be TWEEN 80 and the second hydrophilic surfactant can be KOLLIPHOR RH 40.

### Pharmaceutical Compositions Capable of Forming Micelles Comprising Lipophilic and Hydrophilic Surfactant Systems

In certain embodiments, this disclosure provides pharmaceutical compositions capable of forming micelles comprising estradiol, progesterone, a lipophilic surfactant system, a hydrophilic surfactant system, and, optionally, a terpene. Estradiol and progesterone can be included in the pharmaceutical compositions in the amounts discussed elsewhere herein.

In these embodiments, the lipophilic surfactant system and the hydrophilic surfactant system can have the pharmaceutical compositions and properties described elsewhere herein. As such, and in some embodiments, this disclosure provides pharmaceutical compositions comprising a steroid hormone in the amounts identified elsewhere herein, a lipophilic surfactant system comprising a first lipophilic and second lipophilic surfactant, a hydrophilic surfactant system comprising a first and second hydrophilic surfactant, and an optional terpene.

In some embodiments, the first and second lipophilic surfactants can be first and second partial triglycerides such as CAPMUL 708G and CAPMUL MCM NF, respectively, in the various ratios discussed elsewhere herein. The first hydrophilic surfactants can be TWEEN 80 and the second hydrophilic surfactant can be KOLLIPHOR RH 40 or TPGS 1000. The first and second hydrophilic surfactants can be present in the ratios and quantities described elsewhere herein.

In certain embodiments, the pharmaceutical compositions described in this disclosure can be completely or substantially free of animal oils, vegetable oils, and fractionated vegetable oils. Exemplary excluded animal oils include, but are not limited to, fish liver oils, shark oil, and mink oil. Exemplary excluded fractionated vegetable oils include, but are not limited to, fractionated coconut oils. Exemplary excluded vegetable oils include soy bean oil, safflower seed oil, corn oil, olive oil, cottonseed oil, arachis oil, sunflower seed oil, coconut oil, palm oil, and rape seed oil. In preferred embodiments, the pharmaceutical compositions described in this disclosure are completely or substantially free of all omega-3 free fatty acids and all omega-3 fatty acid esters, including, for example, hexadecatrienoic acid, .alpha.-linolenic acid, stearidonic acid, eicosatrienoic acid, eicosapentaenoic acid, heneicosapentaenoic acid, docosapentenoic acid, docosahexaenoic acid, tetracosapentenoic acid, tetracosahexaenoic acid, or combinations thereof. In preferred embodiments, the pharmaceutical compositions described herein are completely or substantially free of EPA fatty acid esters and DHA fatty acid esters.

### Non-Micelle Forming Pharmaceutical Compositions Comprising a Lipophilic Surfactant System in the Absence of Hydrophilic Surfactants

In certain embodiments, this disclosure provides non-micelle forming pharmaceutical compositions comprising estradiol, progesterone and a lipophilic surfactant system in the absence of hydrophilic surfactants, and, optionally, a terpene. The estradiol and progesterone can be present in the non-micelle forming compositions in amounts discussed elsewhere herein.

In these embodiments, the lipophilic surfactant system can have the pharmaceutical compositions and properties described elsewhere herein. As such, and in some embodiments, this disclosure provides pharmaceutical compositions comprising a estradiol and progesterone in the amounts identified elsewhere herein, a lipophilic surfactant system comprising a first lipophilic and second lipophilic surfactant, and an optional terpene all in the absence of the hydrophilic surfactant system.

In some embodiments, the first and second lipophilic surfactants can be first and second partial triglycerides such as CAPMUL 708G and CAPMUL MCM NF, respectively, in the various ratios discussed elsewhere herein.

In certain embodiments, the non-micelle forming pharmaceutical compositions described in this disclosure can be completely or substantially free of animal oils, vegetable oils, fractionated vegetable oils, all omega-3 free fatty acids, all omega-3 fatty acid esters, EPA fatty acid esters, and DHA fatty acid esters. Exemplary excluded animal oils include, but are not limited to, fish liver oils, shark oil, and mink oil. Exemplary excluded fractionated vegetable oils include, but are not limited to, fractionated coconut oils. Exemplary excluded vegetable oils include soy bean oil, safflower seed oil, corn oil, olive oil, cottonseed oil, arachis oil, sunflower seed oil, coconut oil, palm oil, and rape seed oil. Exemplary excluded omega-3 free fatty acids and omega-3 fatty acid esters, include, for example, hexadecatrienoic acid, α-linolenic acid, stearidonic acid, eicosatrienoic acid, eicosapentaenoic acid, heneicosapentaenoic acid, docosapentenoic acid, docosahexaenoic acid, tetracosapentenoic acid, tetracosahexaenoic acid, combinations thereof, or esters thereof.

### Steroid Hormones

In certain embodiments, the pharmaceutical compositions can comprise from about 0.025 weight percent to about 15 weight percent of a steroid hormone or a combination of steroid hormones. In certain embodiments, the pharmaceutical composition can comprise from about 0.025 weight percent to about 10 weight percent of estradiol and progesterone, from about 1 to about 10 weight percent estradiol and progesterone, about 1 to about 9 weight percent estradiol and progesterone, from about 1 to about 8 weight percent estradiol and progesterone, from about 1 to about 7 weight percent estradiol and progesterone, from about 2 to about 7 weight percent estradiol and progesterone, from about 3 to about 7 weight percent estradiol and progesterone, from about 4 to about 7 weight percent estradiol and progesterone, from about 5 to about 7 weight percent estradiol and progesterone, or about 6 weight percent estradiol and progesterone.

The steroid hormone, and in particular embodiments, estradiol and progesterone, can be partially solubilized (i.e. less than about 80% solubilized), solubilized, or fully solubilized, depending upon the specific components of the composition. In typical embodiments, the estradiol and progesterone are each at least partially solubilized and in certain embodiments, both the estradiol and the progesterone are fully solubilized in the pharmaceutical composition. In some embodiments, the pharmaceutical composition is saturated such that additional steroid hormone, such as progesterone, will not dissolve. In some embodiments, the pharmaceutical composition contains both solubilized and suspended (insoluble) steroid hormone, such as progesterone. That said, and more typically, both the estradiol and the progesterone are each at least about 80%, at least about 85%, at least about 90%, at least about 95%, at least about 99%, or 100% solubilized in the pharmaceutical composition at a given concentration. In certain embodiments, the estradiol and the progesterone are both fully solubilized, i.e., at least about 95 percent solubilized, at least about 98% solubilized, or at least about 99% solubilized as measured according to the methodology described elsewhere herein. However, in other embodiments, the estradiol and the progesterone can be solubilized or only partially solubilized.

In certain embodiments, the bio-identical steroid hormone estradiol can be replaced with estrone, estriol or estrogen analogs. Similarly, the bio-identical steroid hormone progesterone can be replaced with progesterone analogs.

Although the estradiol and progesterone used to formulate the pharmaceutical compositions can have any particle size, in certain embodiments, the estradiol and progesterone can each have an average particle size of less than about 100 microns. In certain embodiments, one or both of the estradiol and the progesterone can be micronized. Without wishing to be bound by any particular theory, it is believed that steroid hormones having a smaller average particle size will be more soluble in the pharmaceutical compositions.

### Terpenes

The pharmaceutical compositions can also include an optional terpene. Terpenes are the primary constituents of the essential oils of many types of plants and flowers and are typically formed directly from one or more isoprene (C₅H₈) units. Terpenes can be naturally occurring or prepared synthetically. Terpenes can be obtained from their natural source, for example, isolated from a natural oil such as citrus oil or orange oil, and optionally purified to be substantially pure, or synthesized chemically.

In certain embodiments, the terpene can be a terpenoid. Examples of terpenes are provided, for example, in Dev et al., “CRC Handbook of Terpenoids: Acyclic, Monocyclic, Bicyclic, Tricyclic, and Tetracyclic Terpenoids” (1989) CRC Press Inc.; Hanson, J. R., Annu. Rep. Prog. Chem., Sect. B: Org. Chem., (1985) 82, 353-375; and Degenhardt et al., Phytochemistry (2009) 70:1621-1637. Each of these references is hereby incorporated by reference in its entirety.

The optional terpene can be linear or cyclic (including aromatic). A cyclic terpene can be a monocyclic terpene or a bicyclic terpene. In a particular embodiment, the cyclic terpene can be a monocyclic terpene. In certain embodiments, the cyclic terpene can be non-aromatic. Examples of cyclic terpenes include, without limitation, limonene (as d-limonene, l-limonene, or a mixture thereof), phellandrene (alpha or beta), camphor, menthol, menthene, carvone, terpinene (alpha, beta, or gamma), terpineol (alpha, beta, or gamma), alpha-ionone, thujone, and derivatives thereof. In certain embodiments, the cyclic terpene is limonene, menthene, menthol, phellandrene, terpinene, or terpineol. In some embodiments, the optional terpene can be d-limonene.

In certain embodiments, when the terpene is present, the terpene can comprise from about 0.5 weight percent to about 10 weight percent of the pharmaceutical composition; from about 1 weight percent to about 10 weight percent of the pharmaceutical composition; from about 2 weight percent to about 9 weight percent of the pharmaceutical composition; from about 3 weight percent to about 8 weight percent of the pharmaceutical composition; from about 4 weight percent to about 8 weight percent of the pharmaceutical composition; from about 5 weight percent to about 7 weight percent of the pharmaceutical composition, or about 6 weight percent of the pharmaceutical composition.

In certain embodiments, the optional terpene is d-limonene and is present in any of the amounts noted above. In other embodiments, the optional terpene is d-limonene and is present at about 6 weight percent of the pharmaceutical composition.

In certain embodiments, the pharmaceutical composition can further include an antioxidant such as α-tocopherol acetate, acetone sodium bisulfite, acetylcysteine, ascorbic acid, ascorbyl palmitate, butylated hydroxyanisole (BHA), butylated hydroxytoluene (BHT), cysteine, cysteine hydrochloride, α-tocopherol, dithiothreitol, monothioglycerol, nordihydroguaiaretic acid, propyl gallate, sodium bisulfite, sodium formaldehyde sulfoxylate, sodium metabisulfite, sodium sulfite, sodium thiosulfate, thiourea, tocopherol, or any combination thereof. In a particular embodiments, the antioxidant is BHT.

The antioxidant can be included in an amount appropriate to inhibit oxidation of any, some, or all of the components of the pharmaceutical composition for a desired period of time. For example, the antioxidant can inhibit oxidation of any of the steroid hormone(s), such as estradiol and/or progesterone, present in the pharmaceutical composition, any of the lipophilic surfactants, any of the hydrophilic surfactants, or the terpene to the extent these components are present in the composition. In certain embodiments, the antioxidant is present to inhibit the oxidation of the terpene, which in certain embodiments, can be d-limonene. In certain embodiments, the BHT is present in the pharmaceutical composition at from about 0.01 to about 0.1 weight percent. In other embodiments, the BHT is present at about 0.03 weight percent.

### Methods of Treating Hormone Deficiencies

In certain embodiments, this disclosure provides methods for treating one or more conditions associated with hormone deficiency in a subject. such as estrogen deficiency and/or progesterone deficiency. The methods comprise orally administering to a subject in need thereof an effective amount of the pharmaceutical composition described herein.

In some embodiments, the condition being treated can be an estrogen deficiency. In some embodiments, the conditions to be treated can be a condition associate with or related to menopause, including hot flashes/flushes, night sweats, sleep disturbances, mood changes, vulvovaginal atrophy, or osteoporosis. Importantly, the progesterone is present in the composition disclosed herein to counteract side effects of estradiol in subjects receiving estradiol therapy.

In some embodiments, the condition being treated can be a progesterone deficiency. In some embodiments, the condition can be endometrial hyperplasia, secondary amenorrhea, hot flashes, night sweats, sleep disturbances, mood changes, or osteoporosis. In some embodiments, progesterone is delivered, together with estradiol, to treat vasomotor symptoms of menopause, including, hot flashes, night sweats, sleep disturbances, mood changes, or osteoporosis. In some embodiments, the progesterone deficiency is menopause. In some embodiments, the pharmaceutical composition disclosed herein can be used to counteract side effects of estradiol in subjects receiving estradiol therapy.

In certain embodiments, the pharmaceutical composition can be administered to a subject in need thereof, such that the subject receives steroid hormone, and in particular embodiments, progesterone, in an amount ranging from about 0.1 mg to about 1 g; about 1 mg to about 600 mg; or about 10 mg to about 500 mg. In certain specific embodiments, the steroid hormone is a combination of estradiol and progesterone.

In certain embodiments, the estradiol can be administered to a subject in need thereof, and in particular a human, using the pharmaceutical compositions in this disclosure so that the subject/human/woman in need thereof receives an amount of estradiol ranging from about 0.01 mg to about 2 mg, and in certain embodiments, about 2 mg, about 1.5 mg, about 1 mg, about 0.75 mg, about 0.5 mg, about 0.25 mg, about 0.2 mg, about 0.15 mg about 0.1 mg, about 0.075 mg, about 0.050 mg, about 0.025 mg, about 0.01 mg, or any range encompassing any of the noted values.

In other embodiments, the progesterone can be administered to a subject in need thereof, and in particular a human, using the pharmaceutical compositions in this disclosure so that the subject/human in need thereof receives an amount of progesterone ranging from about 10 mg to about 500 mg, and in certain embodiments, about 10 mg, about 15 mg, about 20 mg, about 25 mg, 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, or any range encompassing any of the noted values.

In particular embodiments, the amount of progesterone administered per dose using the pharmaceutical composition in this disclosure to a human in need thereof, can range from about 10 mg to about 50 mg or from about 15 mg to about 45 mg. In certain embodiments, the amount of progesterone administered to a subject in need thereof using the pharmaceutical composition of this disclosure can be about 15 mg, about 16 mg, about 17 mg, about 18 mg, about 19 mg, about 20 mg, about 25 mg, about 30 mg, about 31 mg, about 32 mg, about 33 mg, about 34 mg, about 35 mg, about 36 mg, about 37, about 38 mg, about 39 mg, or about 40 mg progesterone. In particular embodiments, a human in need thereof can receive either about 20 mg progesterone or about 36 mg progesterone when the pharmaceutical composition is administered.

In order to receive the desired amount of estradiol and progesterone per dose, the human in need thereof can, in certain embodiments, be administered from about 300 mg to about 2000 mg of the pharmaceutical composition, from about 350 mg to about 1700 mg of the pharmaceutical composition, from about 400 mg to about 1400 mg of the pharmaceutical composition, from about 450 mg to about 1100 mg of the pharmaceutical composition, from about 500 mg to about 800 mg of the pharmaceutical composition, from about 550 mg to about 750 mg of the pharmaceutical composition, from about 575 mg to about 625 mg of the pharmaceutical composition, or about 600 mg of the pharmaceutical formulation. In other embodiments, the human in need thereof can be administered about 300 to about 350 mg of the pharmaceutical composition. In other embodiments, the human in need thereof can be administered about 350 to about 400 mg of the pharmaceutical composition. In other embodiments, the human in need thereof can be administered about 400 to about 450 mg of the pharmaceutical composition. In other embodiments, the human in need thereof can be administered about 450 to about 500 mg of the pharmaceutical composition. In other embodiments, the human in need thereof can be administered about 500 to about 550 mg of the pharmaceutical composition. In other embodiments, the human in need thereof can be administered about 550 to about 600 mg of the pharmaceutical composition. In other embodiments, the human in need thereof can be administered about 600 to about 650 mg of the pharmaceutical composition.

In embodiments wherein the amount of estradiol and progesterone in the composition is about 6 weight percent of the composition and wherein the amounts of estradiol and progesterone to be administered to the human in need thereof are about 0.01 mg to about 2.0 mg estradiol and about 20 mg to about 40 mg progesterone, the amount of the pharmaceutical formulation that can be administered to the human can be about 300 mg to about 600 mg.

In embodiments wherein the amount of estradiol and progesterone in the composition is about 6 weight percent of the composition and wherein the amount of estradiol and progesterone to be administered to the human in need thereof is about 0.01 to about 2.0 mg estradiol and about 30 to about 42 mg progesterone, the amount of the pharmaceutical formulation that can be administered to the human can be about 450 mg to about 600 mg.

These dosages reflect the surprisingly enhanced bioavailability of estradiol and, in particular, progesterone provided by the present pharmaceutical compositions. These compositions provide the opportunity to reduce the amount of estradiol and/or progesterone administered to a human in need thereof relative to currently marketed products such as ESTRACE and/or PROMETRIUM. As discussed elsewhere herein, the PK parameters observed for progesterone when the present pharmaceutical compositions are dosed are highly surprising in view of the known PK parameters associated with PROMETRIUM.

In certain embodiments, the pharmaceutical compositions comprising both estradiol and progesterone can be administered to a human in need thereof in the amounts described above for the treatment of a disease or conditions treatable with estrogen and, in particular, estradiol. Such diseases and conditions include those related to low estrogen levels and, in particular, menopause. Conditions associated with low estrogen levels include hot flashes, hot flushes, night sweats, sleep disturbances, mood changes, vulvovaginal atrophy, or osteoporosis. Conditions associated with menopause include vasomotor symptoms, such as hot flashes/flushes, night sweats and sleep disturbances. Importantly, the progesterone is present in the composition disclosed herein to counteract side effects of estradiol in subjects receiving estradiol therapy.

In certain embodiments, a human can be administered from about 300 mg to about 650 mg of a pharmaceutical compositions described herein to treat a disease or condition associated with low estrogen levels.

In other embodiments, a human can be administered from about 300 mg to about 650 mg of a pharmaceutical compositions described herein to a vasomotor symptom of menopause, such as hot flashes, night sweats and sleep disturbances.

In other embodiments, a human can be administered from about 300 mg to about 650 mg of a pharmaceutical compositions described herein to treat vulvovaginal atrophy.

In other embodiments, a human can be administered from about 300 mg to about 650 mg of a pharmaceutical compositions described herein to treat osteoporosis.

In each of the above described embodiments, a human can be administered about a dose of about 333 mg or about 600 mg of the pharmaceutical composition, such that the human receives about 20 mg or about 36 mg of progesterone per dose of the pharmaceutical composition.

In certain embodiments, the pharmaceutical composition can be administered once daily within in any of the above noted amounts until the disease or condition is treated.

In further embodiments, about 300 mg to about 600 mg of the pharmaceutical composition can be administered once daily to treat the disease or condition associated with low estrogen levels.

In still another embodiment, about 300 mg to about 600 mg of the pharmaceutical composition can be administered once daily to treat the disease or condition associated with menopause, such as a vasomotor symptom of menopause.

In certain embodiments, the amount of pharmaceutical composition administered to a given human subject can be an amount that renders the pharmaceutical composition bioequivalent to ESTRACE and/or PROMETRIUM.

In certain embodiments, the amount of the pharmaceutical composition that is bioequivalent to PROMETRIUM can be from about 300 to about 350 mg of the pharmaceutical composition. In certain embodiments, the pharmaceutical composition can comprise about 6 weight percent progesterone. In still further embodiments, the amount of progesterone administered to the human subject using the present pharmaceutical compositions to achieve bioequivalence to PROMETRIUM can be about 20 mg progesterone.

Although the pharmacokinetic profiles of many progesterone formulations can be affected by whether or not the formulation is taken with food, it has been surprisingly discovered that, in some embodiments, the present pharmaceutical compositions can deliver progesterone consistently both in the presence and absence of food. That is, and surprisingly, in some embodiments, the present pharmaceutical compositions do not show a food effect. This is an extremely beneficial property of certain embodiments of the disclosed pharmaceutical compositions as it allows for less restrictive dosing and increases the likelihood of patient compliance with a given dosing regimen. Lack of a food effect may further reduce both inter- and intra-patient variability when the pharmaceutical compositions of the present disclosure are dosed.

In certain embodiments, the pharmaceutical composition can be administered once daily until the condition is treated.

### Pharmacokinetics and Metabolites

The disclosed pharmaceutical composition can provide enhanced pharmacokinetics versus the currently marketed drugs ESTRACE and PROMETRIUM. For example, in certain embodiments, the pharmaceutical composition can have an AUC₀₋ₜ that is at least about 1.1, at least about 1.2, at least about 1.3, at least about 1.4, at least about 1.5, at least about 1.6, at least about 1.7, at least about 1.8, at least about 1.9, or at least about 2 times greater than ESTRACE and/or PROMETRIUM when the drugs are dosed in the fasting state.

Similarly, in certain embodiments, the pharmaceutical composition can have an Cₘₐₓ that is at least about 1.1, at least about 1.2, at least about 1.3, at least about 1.4, at least about 1.5, at least about 1.6, at least about 1.7, at least about 1.8, at least about 1.9, at least about 2, at least about 2.2, at least about 2.4, at least about 2.6, at least about 2.8, or at least about 3 times greater than ESTRACE and/or PROMETRIUM when the pharmaceutical compositions are dosed in the fasting state.

In certain embodiments, the pharmaceutical composition can have an tₘₐₓ that is at least about 3, at least about 4, at least about 5, at least about 6, at least about 7, at least about 8, at least about 9, at least about 10, at least about 11, at least about 12, at least about 13, at least about 14, at least about 15, at least about 16, or at least about 17 times shorter than ESTRACE and/or PROMETRIUM when the pharmaceutical compositions are dosed in the fasting state. That is, the pharmaceutical composition disclosed herein reaches its Cₘₐₓ considerably earlier than ESTRACE and/or PROMETRIUM.

### Methods for Preparing the Pharmaceutical Compositions

In certain embodiments, the compositions described herein can be prepared according to the following general procedure. In certain embodiments, and in a first step, the steroid hormone, and in particular embodiments, progesterone, can be solubilized in at least one lipophilic surfactant by mixing the steroid hormone with the at least one lipophilic surfactant under mild heating, i.e. from about 35° C. to about 60° C., and in certain embodiments at about 40° C. The mixture can be mixed for an amount of time sufficient to solubilize and uniformly distribute the steroid hormone in the at least one lipophilic surfactant. Typically, the solubilization can be performed in an appropriate vessel, such as an optionally temperature-controlled jacketed stainless steel vessel of the type typically found in medium and large scale formulation manufacturing facilities.

The at least one lipophilic surfactant can have the properties described elsewhere herein and can be added in the amounts specified elsewhere herein. In particular embodiments, the at least one lipophilic surfactant can be a lipophilic surfactant system comprising a first lipophilic surfactant and a second lipophilic surfactant. In some embodiments, the first and second lipophilic surfactants can be first and second partial triglycerides, respectively, wherein the first partial triglyceride is different from the second partial triglyceride.

In some embodiments comprising a first and second partial triglyceride, the first and second partial triglycerides can be independently selected from the group consisting of IMWITOR 988, IMWITOR 742, IMWITOR 308, CAPMUL MCM NF, CAPMUL 708G, with the proviso that the first and second partial triglycerides are different.

In some embodiments, the first and second lipophilic surfactants can be CAPMUL MCM NF and CAPMUL 708G. In certain embodiments, the CAPMUL 708G can be about 90 or about 95 weight percent of the lipophilic surfactant system, with CAPMUL MCM NF, comprising the remaining amount of the surfactant system.

Once the steroid hormone has sufficiently dissolved in the at least one lipophilic surfactant or surfactant system, additional components which can be included in a given composition as specified elsewhere herein, can also be added. For example, in certain embodiments, at least one hydrophilic surfactant can be added to the lipophilic surfactant/steroid hormone mixture in the amounts specified elsewhere herein.

In certain embodiments, the at least one hydrophilic surfactant comprises a hydrophilic surfactant system. In certain embodiments, the hydrophilic surfactant system can comprise a first hydrophilic surfactant and a second hydrophilic surfactant. The first and second hydrophilic surfactants can be selected from any of the suitable hydrophilic surfactants discussed above.

In one embodiments, the first hydrophilic surfactant can be a polyoxyethylene sorbitan fatty acid derivative. In further embodiments, the polyoxyethylene sorbitan fatty acid derivative can be TWEEN 20 (polysorbate 20) or TWEEN 80 (polysorbate 80). In still further embodiments, the first hydrophilic surfactant can be TWEEN 80.

In other embodiments, the second hydrophilic surfactant can be a castor oil or hydrogenated castor oil ethoxylate. In particular embodiments, the castor oil or hydrogenated castor oil ethoxylate can be CREMOPHOR EL, CREMOPHOR RH40, ETOCAS 40, CRODURET 60, or KOLLIPHOR HS 15. In particular embodiments, the second hydrophilic surfactant can be KOLLIPHOR RH 40.

In other embodiments, the second hydrophilic surfactant can be LABRASOL, TPGS 1000, or ascorbyl-6 palmitate. In particular embodiments, the second hydrophilic surfactant can be TPGS 1000.

In certain embodiments, in addition to the at least one hydrophilic surfactant, an antioxidant can also be added. The antioxidant can be added in amounts and embodiments consistent with those disclosed elsewhere herein. In alternative embodiments, the antioxidant can be omitted.

Typically, and when added to a given composition, the various additional components are added with mixing and under mild heating to ensure homogenous distribution of the various components in the composition.

Once the addition of all of the necessary or desired components is complete, the composition can be stirred until it reaches room temperature. Once at room temperature, and when desired, a terpene, such as d-limonene, can be added to the composition in any of the amounts specified elsewhere herein.

The resulting composition, after an optional deaeration process, can then be used as the fill material in the encapsulation process disclosed below.

In another embodiment, the compositions described herein may be prepared by mixing the desired components, exclusive of the optional terpene, at room temperature and subsequently warming the resulting mixture to from about 35° C. to about 60° C., and in certain embodiments to about 40° C. to affect dissolution of the steroid hormone. Following a sufficient amount of stirring to ensure the desired level of dissolution and homogenous distribution of the various components in the composition, the mixture can be cooled to room temperature. After cooling, and as in the alternative embodiment discussed above, a terpene, such as d-limonene, can be added to the composition in any of the amounts specified elsewhere herein.

The resulting composition, after an optional deaeration process, can then be used as the fill material in the encapsulation process disclosed below.

### Encapsulation

Although the pharmaceutical composition can be dosed as a liquid, in certain embodiments, the pharmaceutical composition can be encapsulated in a gelatin capsule, or other similar encapsulated dosage form known to those of skill in the art. The gelatin capsule can be a soft gelatin capsule or a hard gelatin capsule. The hard gelatin capsule can be a two-piece, standard gelatin capsule which typically includes a first capsule portion (i.e., half or bottom) and a second capsule portion (i.e., the other half or top). The soft gelatin capsule can be a two-piece capsule wherein two portions are sealed together or a one-piece, hermetically sealed capsule.

In certain embodiments, the soft gelatin capsule can be a one-piece, hermetically sealed gelatin based capsule which can be made by techniques known to those skilled in the art. In certain embodiments, the gelatin used to form the soft gelatin capsule can include water, gelatin, and a plasticizer to control the softness and flexibility of the capsule. Other additives for use in the gelatin suitable for preparing the soft gelatin capsule, include but are not limited to, flavorants, colorants, and opacifiers.

Soft gelatin capsules can be produced in a known manner, including with a rotary die process in which a molten mass of a gelatin containing the appropriate or necessary additives, is fed from a reservoir onto drums to form two spaced sheets or ribbons of gelatin in a semi-molten state. These ribbons are fed around rollers and brought together at convergent angle into the nip of a pair of roller dies that include opposed die cavities. A liquid fill composition, such as the pharmaceutical composition of this disclosure, can then be fed into the wedge-shaped joinder of the ribbons. The gelatin ribbons are continuously conveyed between the dies, with portions of the fill composition being trapped between the sheets inside the die cavities. The sheets are then pressed together, and severed around each die so that opposed edges of the sheet flow together to form a continuous gelatin sheath around the entrapped liquid composition. The part of the gelatin sheet that is severed from the segments forming the capsules can then be collected for recycling or can be discarded. The resulting soft capsules can then be dried and packaged.

Various gelatin compositions known in the prior art can be used to encapsulate the pharmaceutical composition of this disclosure. For example, suitable gelatin capsules can be prepared from a gelatin mixture comprising from about 30% w/w to about 85% w/w gelatin and in certain embodiments, about 30% w/w to about 50% w/w; about 15% w/w to about 40% w/w of one or more plasticizer; and from 25% w/w to about 50% w/w of water. In certain embodiments, the gelatin will have a bloom in the rage of about 150 to about 275, and can be Type A or B gelatins or a mixture thereof.

Examples of suitable Type A gelatin include without limitation acid bone gelatin. Examples of suitable Type B gelatin include without limitation lime bone gelatin.

Suitable gelatin plasticizers are well known to those of ordinary skill in the art and include, but are not limited to, polyhydric alcohols such as sorbitol, glycerin, mannitol, xylitol, maltitol, and sorbitan; dialkylphthalates; lower alkyl citrates wherein the lower alkyl has 1-6 carbon atoms; glycols and polyglycols including polyethylene glycols with a molecular weight range of about 200 to about 2,000, methoxyl-propylene-glycol, and 1,2-propylene glycol; esters of polyhydroxy-alcohols such as mono-, di-, and tri-acetate of glycerol; ricinoleic acid and esters thereof; and mixtures of the above. The gelatin composition can also contain other ingredients including, but not limited to, taste modifiers, coloring agents, opacifiers, and moisture retaining agents.

### EXAMPLES

The pharmaceutical composition described herein is now further detailed with reference to the following examples. These examples are provided for the purpose of illustration only and the embodiments described herein should in no way be construed as being limited to these examples. Rather, the embodiments should be construed to encompass any and all variations which become evident as a result of the teaching provided herein.

### Example 1: Pharmaceutical Compositions

Pharmaceutical compositions having both estradiol and progesterone as well as the ingredients shown in Tables 1-3 are readily prepared by combining the ingredients using standard preparatory techniques.

TABLE 1
Estradiol and Progesterone Fill Formulas (all values presented in mg/g)

| | Pharma. Composition | | | | | | |
|---|---|---|---|---|---|---|---|
| Component | A1 | B1 | C1 | D1 | E1 | F1 | G1 |
| CAPMUL 708G | 761.06 | 723.01 | 723.01 | 761.06 | 834.62 | — | 751.16 |
| CAPMUL | 84.56 | 80.33 | 80.33 | 84.56 | — | 834.62 | 83.46 |
| MCM, NF | | | | | | | |
| Ultra High | — | 42.28 | 42.28 | — | — | — | — |
| Purity d- | | | | | | | |
| limonene | | | | | | | |
| BHT | 0.28 | 0.28 | 0.28 | 0.28 | — | — | — |
| Estradiol | 2.00 | 2.00 | 2.00 | 2.00 | 2.00 | 2.00 | 2.00 |
| Progesterone | 60.13 | 60.13 | 60.13 | 60.13 | 72.64 | 72.64 | 72.64 |
| Polysorbate 80 | 70.47 | 70.47 | 46.98 | 46.98 | 69.55 | 69.55 | 69.55 |
| TPGS 1000 | 21.49 | 21.49 | — | — | 21.18 | 21.18 | 21.18 |
| KOLLIPHOR | — | — | 44.98 | 44.98 | — | — | — |
| RH 40 | | | | | | | |

TABLE 2
Estradiol and Progesterone Fill Formulas (all values presented in mg/g)

| | Pharma. Composition | | | | | | |
|---|---|---|---|---|---|---|---|
| Component | H1 | I1 | J1 | K1 | L1 | M1 | N1 |
| CAPMUL 708G | 761.06 | 723.01 | 723.01 | 761.06 | 834.62 | — | 751.16 |
| CAPMUL | 84.56 | 80.33 | 80.33 | 84.56 | — | 834.62 | 83.46 |
| MCM, NF | | | | | | | |
| Ultra High | — | 42.28 | 42.28 | — | — | — | — |
| Purity d- | | | | | | | |
| limonene | | | | | | | |
| BHT | 0.28 | 0.28 | 0.28 | 0.28 | — | — | — |
| Estradiol | 0.50 | 0.50 | 0.50 | 0.50 | 0.50 | 0.50 | 0.50 |
| Progesterone | 60.13 | 60.13 | 60.13 | 60.13 | 72.64 | 72.64 | 72.64 |
| Polysorbate 80 | 70.47 | 70.47 | 46.98 | 46.98 | 69.55 | 69.55 | 69.55 |
| TPGS 1000 | 22.99 | 22.99 | — | — | 22.68 | 22.68 | 22.68 |
| KOLLIPHOR | — | — | 46.48 | 46.48 | — | — | — |
| RH 40 | | | | | | | |

TABLE 3
Estradiol and Progesterone Fill Formulas (all values presented in mg/g)

| | Pharma. Composition | | | | | | |
|---|---|---|---|---|---|---|---|
| Component | O1 | P1 | Q1 | R1 | S1 | T1 | U1 |
| CAPMUL 708G | 761.06 | 723.01 | 723.01 | 761.06 | 834.62 | — | 751.16 |
| CAPMUL | 84.56 | 80.33 | 80.33 | 84.56 | — | 834.62 | 83.416 |
| MCM, NF | | | | | | | |
| Ultra High | — | 42.28 | 42.28 | — | — | — | — |
| Purity d- | | | | | | | |
| limonene | | | | | | | |
| BHT | 0.28 | 0.28 | 0.28 | 0.28 | — | — | — |
| Estradiol | 0.20 | 0.20 | 0.20 | 0.20 | 0.20 | 0.20 | 0.20 |
| Progesterone | 60.13 | 60.13 | 60.13 | 60.13 | 72.64 | 72.64 | 72.64 |
| Polysorbate 80 | 70.47 | 70.47 | 46.98 | 46.98 | 69.55 | 69.55 | 69.55 |
| TPGS 1000 | 23.29 | 23.29 | — | — | 22.02 | 22.02 | 22.02 |
| KOLLIPHOR | — | — | 46.78 | 46.78 | — | — | — |
| RH 40 | | | | | | | |

### Example 2: Pharmaceutical Compositions

Pharmaceutical compositions having the ingredients shown in Table 4 were prepared by combining the ingredients using standard preparatory techniques.

TABLE 4
Solubilized Progesterone Fill Formulas (all values presented in mg/g)

| | Pharma. Composition | | | | | | |
|---|---|---|---|---|---|---|---|
| Component | A | B | C | D | E | F | G |
| Capmul 708G | 761.06 | 723.01 | 723.01 | 761.06 | 834.62 | — | 751.16 |
| Capmul MCM, | 84.56 | 80.33 | 80.33 | 84.56 | — | 834.62 | 83.46 |
| NF | | | | | | | |
| Ultra High | — | 42.28 | 42.28 | — | — | — | — |
| Purity d-limonene | | | | | | | |
| BHT | 0.28 | 0.28 | 0.28 | 0.28 | — | — | — |
| Progesterone | 60.13 | 60.13 | 60.13 | 60.13 | 72.64 | 72.64 | 72.64 |
| Polysorbate 80 | 70.47 | 70.47 | 46.98 | 46.98 | 69.55 | 69.55 | 69.55 |
| TPGS 1000 | 23.49 | 23.49 | — | — | 23.18 | 23.18 | 23.18 |
| Kolliphor RH | — | — | 46.98 | 46.98 | — | — | — |
| 40 | | | | | | | |

### Example 3: Particle Size Analysis

Average particle sizes for each of Pharmaceutical Compositions A, B, C, and D as disclosed in Example 2 were measured using a DELSA Nano photon correlation spectrometer. Approximately 0.5 g of a given sample was diluted with 55 mL of filtered deionized water and the mean size of the resulting particle and the zeta potential was calculated as set forth in Table 5. A similar particle size analysis can be performed for Pharmaceutical Compositions A1-U1 of Tables 1-3 of Example 1, which pharmaceutical compositions contain both estradiol and progesterone.

TABLE 5

| Pharmaceutical | Mean size (nm) ± | Zeta Potential |
|---|---|---|
| composition | Std. Dev. | (mV) |
| A | 301.6 ± 164.4 | −16.89 |
| B | 678.2 ± 698.6 | −16.87 |
| C | 575.2 ± 604.8 | −20.63 |
| D | 156.3 ± 52.2 | −18.37 |

### Example 4: Oral Bioavailability in Rats

Oral bioavailability of the pharmaceutical compositions were assessed in male Sprague-Dawley rats. According to the protocol, 30 male rats were divided into 6 groups of 5 rats each. The rats were then treated with one of the pharmaceutical compositions discussed in Example 2 (Compositions A, B, C, and D), Pharmaceutical Composition H (a non-micelle forming, fully-solubilized progesterone pharmaceutical composition within the scope of this disclosure described more fully in Table 6), or PROMETRIUM according to the schedule shown in Table 7.

TABLE 6
Pharmaceutical Composition H

| Component | Chemical Name | Quantity (mg/g) |
|---|---|---|
| CAPMUL 708G | Glyceryl | 846 |
| | Caprylate | |
| Capmul | Caprylic/capric | 94 |
| MCM, NF | mono/diglycerides | |
| Progesterone | API | 60 |

TABLE 7

| Study | |
|---|---|
| Day | Event |
| −4 | Animals were transferred to surgery facility and were |
| | group/gang housed. |
| −3 | Animals were observed. |
| −2 | Animals were observed. |
| −1 | Animals were fitted with jugular vein catheters (vaporized |
| | isoflurane anesthesia) and treated with analgesics as per |
| | HLA SOP 7.168. The animals were fasted for 12 hours starting |
| | at 8:00 PM. |
| 0 | Gavage capsules were filled with 20 μl of compound per |
| | capsule. Baseline plasma samples were collected, the animals |
| | received compound via capsule gavage, and additional plasma |
| | samples were taken at 10, 20, 40, 60, 90, 120, 180, and 240 |
| | minutes post dosing. The animals were observed. Frozen plasma |
| | samples were shipped on dry ice for analysis. |

Although PROMETRIUM was dosed in a capsule filled with 20 μL of the PROMETRIUM formulation, the PROMETRIUM capsule contained at least 6 times as much progesterone (400 mg/g formulation) as the test pharmaceutical compositions (60 mg/g composition) due to the way in which PROMETRIUM is formulated.

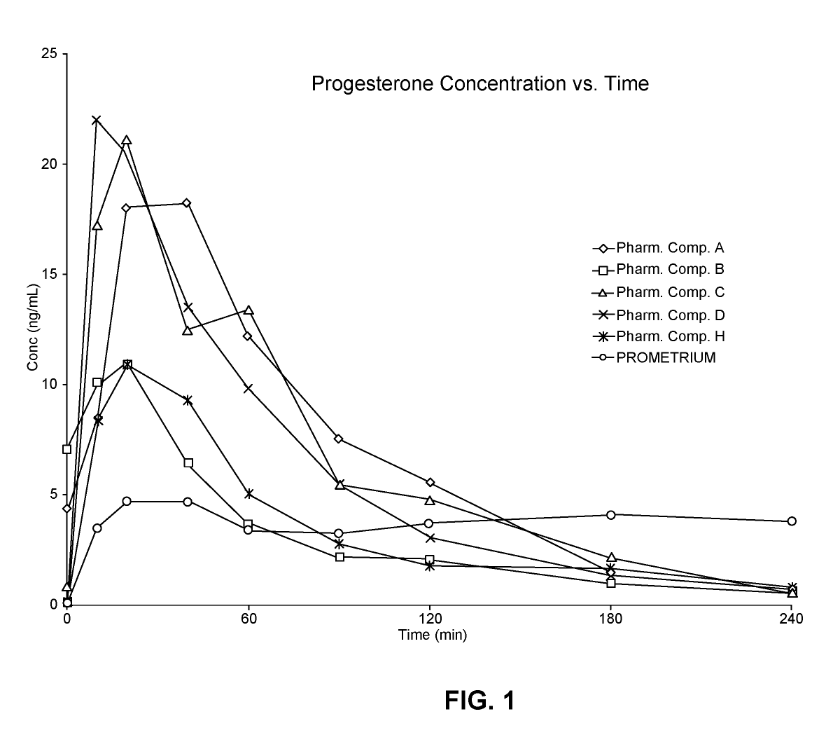

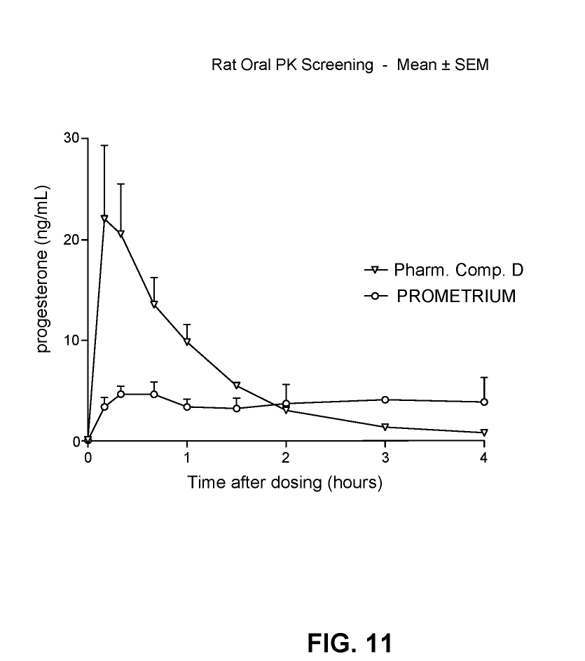

The frozen plasma samples were then analyzed and the data plotted. The results are shown in FIGS. 1 (linear-linear) and 2 (log-linear). Both figures show that test Pharmaceutical Compositions A, C, and D performed better than Pharmaceutical Composition H and PROMETRIUM. FIG. 11 shows the performance of Pharmaceutical Composition D and PROMETRIUM, as both shown in FIG. 1, in the absence of the other tested formulations.

The PK parameters observed (+/− standard deviation) are shown in Table 8.

TABLE 8
Non-Normalized Progesterone PK Data

| | A | B | C | D | H | PROMETRIUM |
|---|---|---|---|---|---|---|
| Cₘₐₓ | 20.8 ± 12.8 | 13 ± 9 | 24.7 ± 11.2 | 23.5 ± 15.3 | 13.1 ± 6.9 | 6.9 ± 4.0 |
| (ng/mL) | | | | | | |
| tₘₐₓ (hr) | 0.467 ± 0.183 | 0.167 ± 0.167 | 0.4 ± 0.346 | 0.333 ± 0.204 | 0.367 ± 0.183 | 2.2 ± 1.609 |
| AUC₀₋ₜ | 27.3 ± 23.0 | 12.5 ± 9.2 | 26.2 ± 11.9 | 24.2 ± 8.8 | 14.1 ± 7.6 | 15.1 ± 8.4 |
| (ng-hr/mL) | | | | | | |
| AUC_{0-∞} | 28.0 ± 23.4 | 14.1 ± 11.1 | 27.1 ± 11.7 | 25.5 ± 8.2 | 15.6 ± 8.5 | 18.9 ± 13.6 |
| (ng-hr/mL) | | | | | | |

Despite containing significantly less progesterone than PROMETRIUM, each of the test pharmaceutical compositions provided a higher Cₘₐₓ (greater than 10-fold) and AUC₀₋ₜ (4.5 to 10-fold—except for Pharmaceutical Composition HH, which showed an AUC similar to PROMETRIUM), when normalized to a standard 1 mg dose than was observed for PROMETRIUM. Each of the test pharmaceutical compositions had a higher Cₘₐₓ and shorter tₘₐₓ than PROMETRIUM, suggesting more rapid absorption.

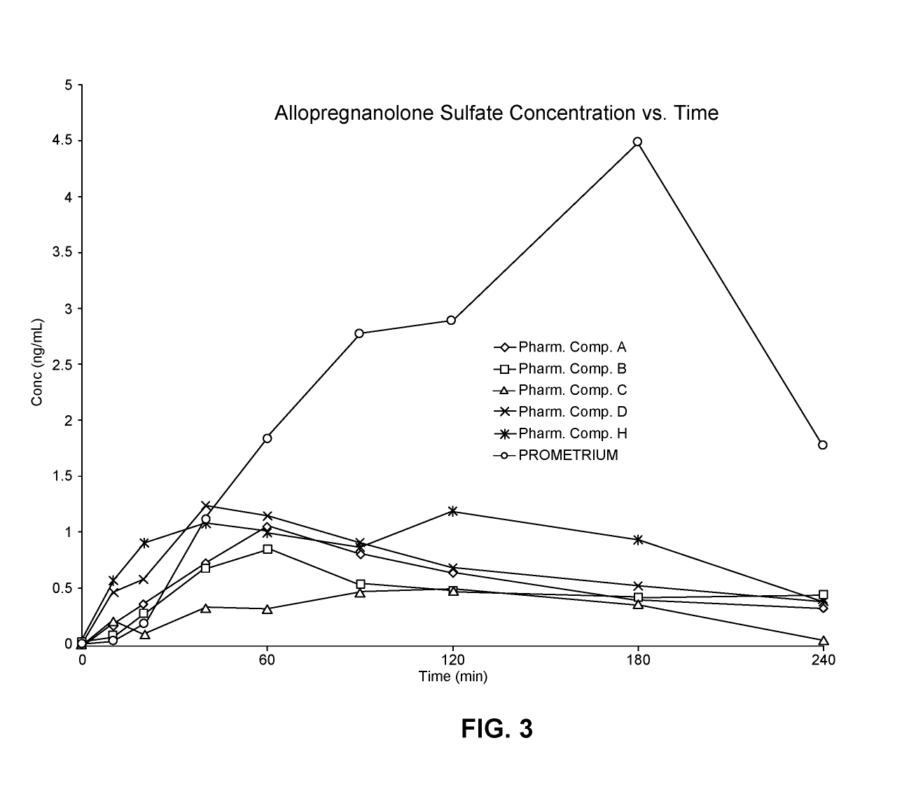

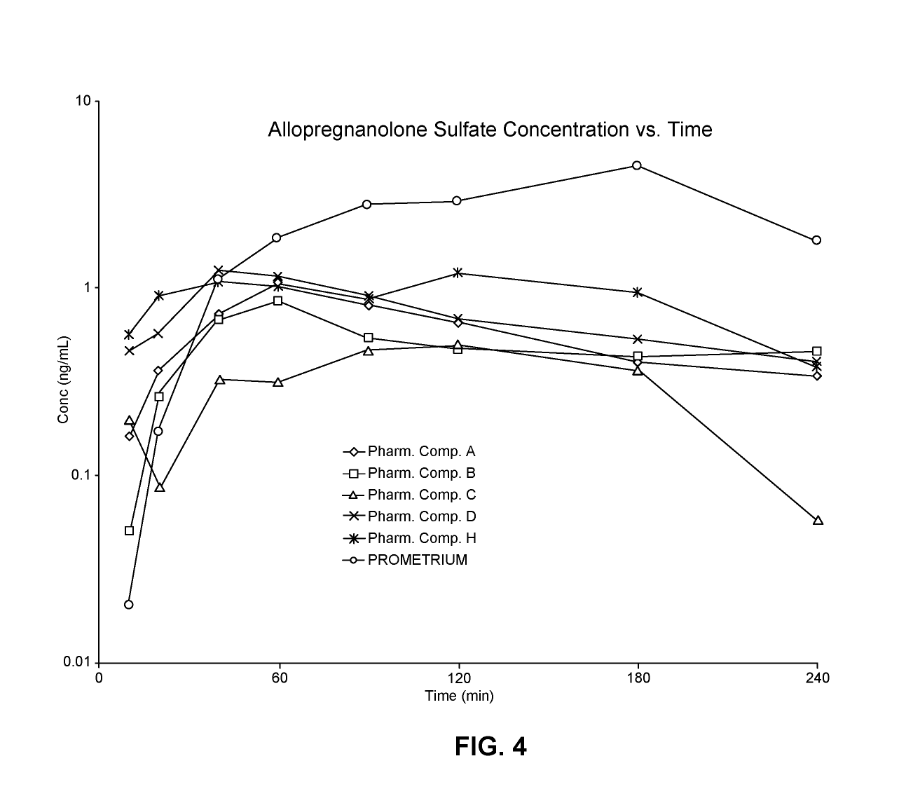

In addition, the relative amount of a down-stream metabolite of progesterone (allopregnanolone sulfate) was much higher in rats dosed with PROMETRIUM (AUC approximately 90% of the AUC for progesterone) than with the test pharmaceutical compositions (approximately 6-15%). Allopregnolone is believed to be associated with somnolence side effect in humans. In certain embodiments, Pharmaceutical Compositions A, B, C, or D can be administered to reduce or eliminate a somnolence side effect in patients needing progesterone therapy. See, FIGS. 3 and 4.

Each of test pharmaceutical compositions also provided faster onset of action than PROMETRIUM by over and hour and a half. In certain embodiments, a faster onset of action demonstrates the improved bioavailability over currently available hormone formulations. Given the vast difference in progesterone concentration between PROMETRIUM and the described pharmaceutical compositions, these results are highly surprising and unexpected.

### Example 5: Food Effect on Oral Absorption

According to the protocol, 56 male rates were divided into 8 groups of 7 rats each. Each group was given one of three test pharmaceutical compositions or PROMETRIUM as set forth in Table 9 according to the schedule shown in Table 10. Animals in “Fed” groups were presented with a pre-weighed amount of food 15 minutes prior to receiving a given pharmaceutical composition. The food was removed 45 minutes after dosing and weighed to calculate average consumption per animal. Animals in fasted groups received food approximately 4 hours after dosing.

TABLE 9

| | Pharmaceutical | |
|---|---|---|
| Group | Composition | Fed/Fasted |
| 1 | PROMETRIUM | Fasted |
| 2 | PROMETRIUM | Fed |
| 3 | D | Fasted |
| 4 | D | Fed |
| 5 | A | Fasted |
| 6 | A | Fed |
| 7 | C | Fasted |
| 8 | C | Fed |

TABLE 10

| Study | |
|---|---|
| Day | Event |
| −4 | Animals were transferred to surgery facility and were |
| | group/gang housed. |
| −3 | Animals were observed. |
| −2 | Animals were observed. |
| −1 | Animals were fitted with jugular vein catheters (vaporized |
| | isoflurane anesthesia) and treated with analgesics as per |
| | HLA SOP 7.168. The animals were fasted for 16 hours |
| | starting at 4:00 PM. |
| 0 | Gavage capsules were filled with 20 μl of composition per |
| | capsule. The Animals were either fed or fasted, as noted |
| | above, and given compositions via capsule gavage. Plasma |
| | samples were taken at 10, 20, 40, 60, 90, 120, 180, and 240 |
| | minutes post dosing. The animals were observed. Frozen plasma |
| | samples were shipped on dry ice for analysis. |

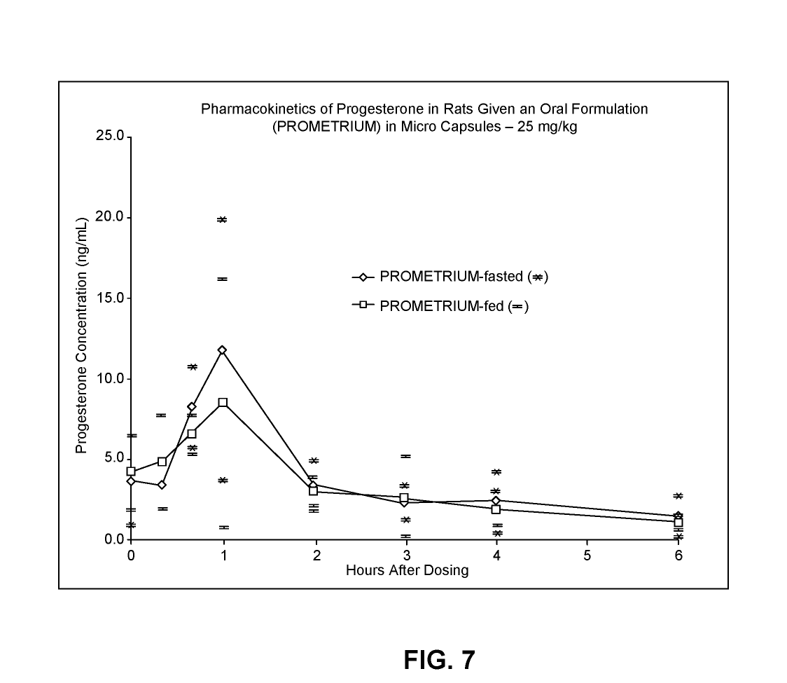

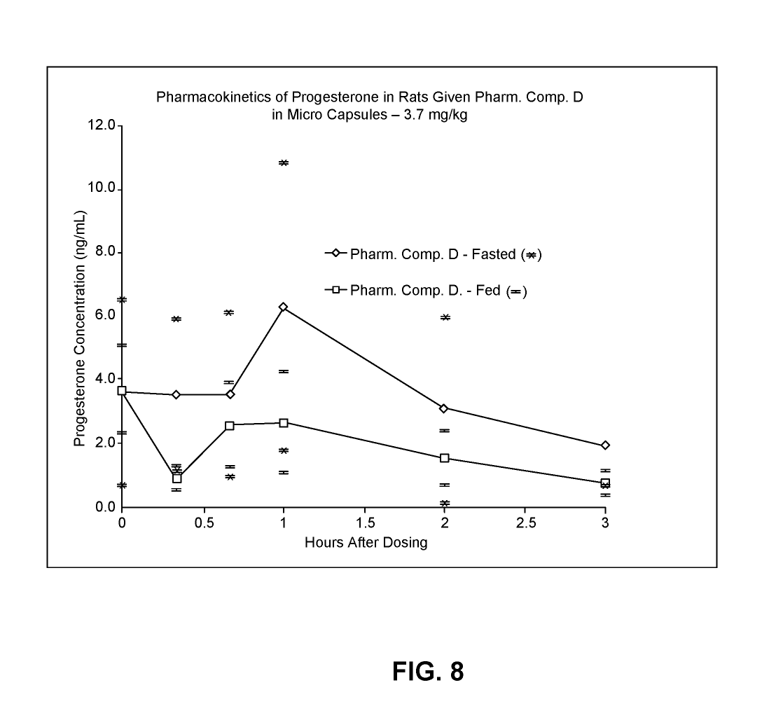

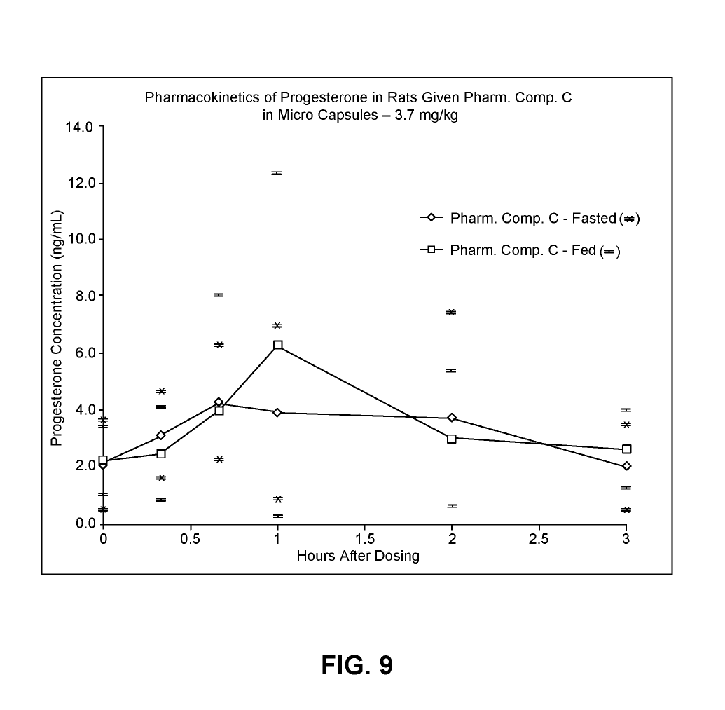

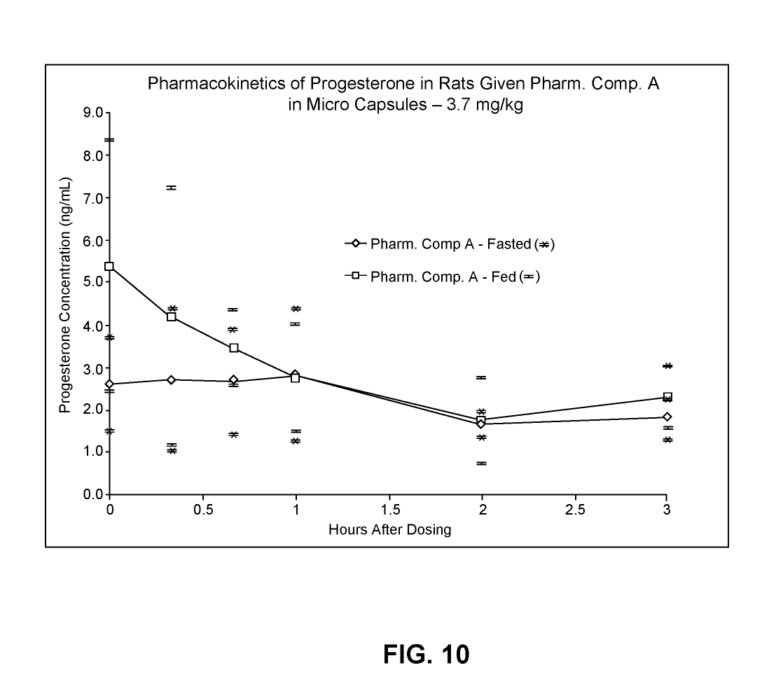

The results of this study are show in FIGS. 7, 8, 9, and 10 and show that there was no clear food effect on the pharmacokinetics of any of the pharmaceutical compositions, but dose-normalized progesterone exposure for the test pharmaceutical compositions was approximately 5-fold higher for Cₘₐₓ and 3-fold higher for AUC₀₋ₜ than for PROMETRIUM.

In certain embodiments, the Cₘₐₓ and AUC₀₋ₜ differences between the test compositions and PROMETRIUM are surprising given that PROMETRIUM contains about 400 mg progesterone per gram of formulation, whereas the test pharmaceutical compositions contain 60 mg progesterone per gram of formulation (i.e. about 6 weight percent). In view of this significant difference in the amount of available progesterone when both compositions were dosed at equal volumes (i.e. 20 μl), a person of ordinary skill in the art would not have predicted that the present pharmaceutical composition would enhanced oral bioavailability versus PROMETRIUM.

The breadth and scope of the present invention should not be limited by any of the above-described exemplary embodiments, but should be defined only in accordance with the following claims and their equivalents.

All patents, patent applications, and other references noted or referenced in this application are hereby incorporated by reference in their entirety.

1. A pharmaceutical composition suitable for administering estradiol and progesterone to a subject in need thereof, the pharmaceutical composition comprising estradiol, progesterone, a lipophilic surfactant system comprising a first lipophilic surfactant and a second lipophilic surfactant, wherein the first and second lipophilic surfactants are different from each other, a hydrophilic surfactant system comprising first and second hydrophilic surfactants, and an optional terpene, wherein the pharmaceutical composition is completely or substantially free of fractionated vegetable oils.

2. The pharmaceutical composition of claim 1, wherein the first lipophilic surfactant is a first partial triglyceride.

3. The pharmaceutical composition of claim 2, wherein the second lipophilic surfactant is a second partial triglyceride.

4. The pharmaceutical composition of claim 3, wherein the first and second partial triglycerides are selected from the group consisting of IMWITOR 988, IMWITOR 742, IMWITOR 308, CAPMUL MCM NF, CAPMUL 708G, and glyceryl dilaurate.

5. The pharmaceutical composition of claim 4, wherein the first partial triglyceride is CAPMUL MCM NF and the second partial triglyceride is CAPMUL 708G.

6. The pharmaceutical composition of claim 5, wherein the first hydrophilic surfactant is a polyoxyethylene sorbitan fatty acid derivative.

7. The pharmaceutical composition of claim 6, wherein the polyoxyethylene sorbitan fatty acid derivative is TWEEN 20 or TWEEN 80.

8. The pharmaceutical composition of claim 6, wherein the second hydrophilic surfactant is a castor oil or hydrogenated castor oil ethoxylate.

9. The pharmaceutical composition of claim 8, wherein the castor oil or hydrogenated castor oil ethoxylate is CREMOPHOR EL, CREMOPHOR RH40, ETOCAS 40, CRODURET 60, or KOLLIPHOR HS 15.

10. The pharmaceutical composition of claim 9, wherein the castor oil or hydrogenated castor oil ethoxylate is CREMOPHOR RH40.

11. The pharmaceutical composition of claim 1, wherein the second hydrophilic surfactant is LABRASOL, TPGS, or ascorbyl-6 palmitate.

12. The pharmaceutical composition of claim 11, wherein the second hydrophilic surfactant is TPGS.

13. The pharmaceutical composition of claim 1, wherein the terpene is not optional and is selected from the group consisting of d-limonene, menthene, menthol, phellandrene, terpinene, or terpineol.

14. The pharmaceutical composition of claim 13, wherein the terpene is d-limonene.

15. The pharmaceutical composition of claim 1, wherein the estradiol and the progesterone are both solubilized.

16. The pharmaceutical composition of claim 1, wherein the estradiol and the progesterone are both fully solubilized.

17. The pharmaceutical composition of claim 1, wherein the pharmaceutical composition forms micelles.

18.-35. (canceled)

36. A method of treating a symptom of menopause, the method comprising administering to a subject in need thereof a pharmaceutical composition according to claim 1.

37. The method of claim 36, wherein the symptom of menopause is selected from the group consisting of hot flushes, hot flashes, vaginal dryness, night sweats, urinary tract infections, loss of libido, heart disease, or osteoporosis.

38.-39. (canceled)

40. A pharmaceutical composition suitable for administering estradiol and progesterone to a subject in need thereof, the pharmaceutical composition comprising estradiol, progesterone, a lipophilic surfactant system comprising a first lipophilic surfactant and a second lipophilic surfactant, wherein the first and second lipophilic surfactants are different from each other, a hydrophilic surfactant system comprising first and second hydrophilic surfactants, and an optional terpene, wherein the pharmaceutical composition is completely or substantially free of fractionated vegetable oils, wherein both the estradiol and the progesterone are solubilized in the pharmaceutical composition, and wherein the pharmaceutical composition forms micelles.

41.-43. (canceled)
